(19) United States
(12) Patent Application Publication
(10) Pub. No.: US 2004/0181344 A1
(43) Pub. Date: Sep. 16, 2004

(54) Systems and methods for providing diagnostic services

(75) Inventors: Gregory Stephanopoulos, Winchester, MA (US); Ilias Alevizos, Athens (GR); Jatin Misra, Cambridge, MA (US)

Correspondence Address: ROPES & GRAY LLP, ONE INTERNATIONAL PLACE, BOSTON, MA, 02110-2624, US

(73) Assignee: Massachusetts Institute of Technology, Cambridge, MA

(21) Appl. No.: 10/716,825

(22) Filed: Nov. 18, 2003

Related U.S. Application Data
(63) Continuation-in-part of application No. 10/060,048, filed on Jan. 29, 2002.
(60) Provisional application No. 60/427,265, filed on Nov. 18, 2002.

(51) Int. Cl. C12Q 1/68; G06F 19/00; G01N 33/48; G01N 33/50

(52) U.S. Cl. 702/20; 435/6

(57) ABSTRACT

Disclosed are diagnostic methods for oral diseases such as oral cancer. In the methods, expression data of a plurality of genes associated with an oral disease is examined and compared to signature expression profile indicative of an oral disease. Also disclosed are systems, methods and kits for allowing a dentist to provide for detection of oral disease at the point of patient care.

### APPLICATION INFORMATION

This application claims priority to U.S. Application No. 60/427,265, filed on Nov. 18, 2002, and U.S. application Ser. No. 10/060,048, filed on Jan. 29, 2002, both of which are incorporated in their entireties by reference herein.

### FUNDING

Work described herein was funded, in whole or in part, by Grant No. DE-FG02-94ER-14487 and DE-FG02-99ER-15015 from the Engineering Research Program of the Office of Basic Energy Science at the Department of Energy, and by Grant No. 1-RO1-DK58533-01 from NIH. The United States government has certain rights in the invention.

### BACKGROUND OF THE INVENTION

Oral cavity cancer is the sixth most common cancer in the United States. It is newly diagnosed in about 31,000 Americans each year and 350,000 people worldwide. One patient dies from oral cancer every hour in the U.S. alone.

Cancers of the mouth present in various forms. Any persistent white patch must be regarded as being suspicious. Additionally, velvety red patches—particularly those with white speckles—should be areas of concern. Finally, any non-healing ulcer (erosion) merits evaluation. More often than not, these areas are painless. The tongue is the most common site of oral cancer. Typically, the side of the tongue (farthest back in the mouth) is involved. The floor of the mouth (that area beneath the tongue) is next in order of frequency followed by the insides of the cheeks with involvement of other areas showing a lesser incidence.

Oral squamous cell carcinoma, for example, has been linked to excessive cigarette smoking and alcohol abuse, both individually and in combination. Other factors associated with oral cancer include poor dental hygiene and malfitting dentures or broken teeth that cause chronic mucosal irritation. Occupational hazards include chronic dust exposure among woodworkers, which has been associated with cancer of the nasopharynx, and exposure to nickel compounds, which increases the risk of paranasal sinus cancers.

About 90% of oral cancers are detected in only a few high-risk sites; the floor of the mouth, the ventrolateral aspect of the tongue, and the soft palate complex. Buccal and labial vestibular carcinoma should be considered in people who use smokeless tobacco.

Early, asymptomatic oral cancer appears most often as a red (erythroplastic) lesion. Squamous cell carcinoma, not diagnosed in its earliest stages appears later as a deep ulcer with smooth, indurated, rolled margins, fixed to deeper tissues. Biopsy is necessary to diagnose carcinoma.

Squamous cell carcinomas are often diagnosed early because such cancers lead to local symptoms such as pain, hoarseness, and difficulty in swallowing. In many cases, however, diagnosis is delayed because local symptoms or pain from nerve involvement does not occur until a large primary tumor develops. In such cases, regional nodal metastases may be the initial manifestation. Distant metastases rarely occur without locally advanced primary disease or nodal involvement.

Patients with oral cancer benefit from vigilant monitoring, early detection and intervention. However, oral cancer can spread through its early stages without detection as most people are unfamiliar with the disease and its symptoms. Accordingly, there is a need in the art for an oral cancer detection and diagnostic process that increases monitoring and early detection.

### SUMMARY OF THE INVENTION

In one aspect, systems and methods described herein provide for detection and diagnosis of oral cancer. According to one method, cells are obtained from a patient and the expression levels of a plurality of genes associated with an oral cancer is determined to generate a test expression profile. This test expression profile is then compared to a signature expression profile of oral cancer, as well as an expression profile of the same set of genes in a healthy subject (“control expression profile”). If the test expression profile from a patient substantially matches the signature expression profile of oral cancer, then the patient is highly likely to have oral cancer.

As described herein, the determination may be made by any suitable means, and the invention is not limited to any particular assay. For purpose of illustration, it will be noted that the determination of gene expression may be made through Northern blot analysis, reverse transcription-polymerase chain reaction (RT-PCR), in situ hybridization, immunoprecipitation, Western blot hybridization, or immunohistochemistry. Additionally, mircoarray analysis may be performed to identify the relative expression of certain proteins and combinations of proteins.

In another aspect, disclosed herein are desktop devices that may be employed at the point of sample collection to provide facile methods for point-of-care diagnostic analysis. The desktop systems that may be employed at the point of sample collection for the purpose of determining whether a patient has, or may have, a particular condition. The desktop systems can include sample collection devices, diagnostic systems that will prepare a sample of biological material for processing by an assay, and will then perform the assay and present the results to the system operator.

In a further aspect, disclosed herein are methods for conducting a business wherein samples from a patient are taken and processed to determine at the point of care whether the patient has a particular condition or indication. In one particular practice, disclosed herein is a method for a dentist to employ a desktop diagnostic system that can process a sample of biological material taken from a dental patient, such as tissue scrape or saliva, and process that sample to determine whether the patient has a particular oral disease, for example, oral cancer. In operation the dentist can collect a saliva and/or cellular sample from the patient, deliver the sample to the desktop machine and the desktop machine can process the sample, optionally in real time, to make a determination as to whether the sample tests positive for an oral disease such as oral cancer. The test results may be provided to the patient at the point-of-care thereby providing immediate diagnosis for initiating treatment procedures for the patient. The method may further comprise obtaining authorization representative of insurance coverage. Either the patient or the medical professional carrying out the methods of the invention may also select a test for an oral disease as a function of insurance coverage, requesting insurance reimbursement for the test and/or generating a medical record representative of the test and result.

In a further aspect, disclosed herein are oral disease detection kits. The kits may include sample collection devices and sample delivery devices adapted for use with the diagnostic system described herein to examine the samples for an indication of an oral disease, such as oral cancer.

### BRIEF DESCRIPTION OF THE DRAWINGS

The foregoing and other objects and advantages of the invention will be appreciated more fully from the following further description thereof, with reference to the accompanying drawings wherein;

FIG. 1 depicts schematically the structure of a system according to the invention.

FIG. 2 depicts one substrate suitable for use with the system of FIG. 1.

FIG. 3 depicts schematically the structure of a collection device for oral cancer detection.

FIG. 4 shows a comparison of two idealized gene expression distributions.

FIG. 5 depicts discriminatory gene selection. (a) shows F statistic transformed from Wilk's lambda. (b) shows error rates estimated by LOOCV.

### DETAILED DESCRIPTION OF CERTAIN ILLUSTRATED EMBODIMENTS

Applicants have discovered a set of genes that are differentially expressed in oral cancer cells versus normal cells. Applicants have shown that the expression profile of this set of genes is indicative of oral cancer, and as such, constitutes a signature expression profile of oral cancer. Thus, measuring expression levels of these genes in a sample cell population allows for the type and tumor stage of the cells in the sample to be determined.

These differentially expressed genes are collectively referred to herein as marker genes. The corresponding gene products are referred to as “marker proteins” or “marker polypeptide”. The marker genes for oral cancer include urokinase plasminogen activator, oncofetal trophoblast glycoprotein, cathepsin L, Wilms tumor related protein, FAT, GRO2, AML1, heat shock protein 90, crystallin alpha-B, aldehyde dehydrogenase-9, aldehyde dehydrogenase-10, carboxylesterase-2, cytochrome p450 and others shown in Table 1.

The subject methods, systems and kits can be used to detect an oral disease such as oral cancer. For this purpose, biological samples from patients can include oral tissue (including epithelial and mucosal tissues), cell scrapes from oral tissue and/or saliva samples.

The subject methods, systems and kits can be additionally used with a variety of other biological samples, i.e., tissues, cells and/or bodily fluids taken from any part of the body, so long as the biological material is suspected of containing the analyte of interest. For example, suitable tissues for use in the present method include, without limitation, adrenal, bladder, bone marrow, brain, breast, cardiac, colon, esopliageal, intestinal, kidney, liver, pulmonary, lymph node, nerve, ovarian, pancreatic, prostatic, skeletal (striated) muscle, smooth muscle, spleen, stomach, testicular, tonsil, tracheal and uterine tissue. Furthermore, cells taken from these same tissues are appropriate for the techniques described herein. Additionally, cells may be obtained from cell-containing fluids, such as peripheral blood, lymph fluid, ascites, serous fluid, pleural effusion, sputum, cerebrospinal fluid, lacrimal fluid, stool or urine. Other embodiments are suitable for analyzing bone marrow aspirates, bone marrow biopsies, lymph node aspirates, lymph node biopsies, fine needle aspirates, or other organ tissue biopsies.

I. Sample Preparation

In some instances, the biological material must be preserved or “fixed”. Although many methods are known in the art for fixing biological samples, it is preferred that the biological sample is fixed with formaldehyde. For example, the biological material maybe combined with a 4% formaldehyde solution for 30 minutes on ice. Alternatively, other fixatives, e.g., alcohol, may also be employed.

When used for detecting changes in expression levels or genetic polymorphisms, it will generally be preferred that the patient sample is processed to render the endogenous amenable to hybridization with detection probes, or to generate alternative nucleic acids species (such as reverse transcription of mRNA to produce cDNA), or to amplify the nucleic acid analytes to improve detection.

Where the analyte to be detected (directly or indirectly) is mRNA, there are a variety of well known techniques for processing patient samples and isolating mRNA in a form suitable for further use in the subject method. Merely to illustrate, the PolyA-tract system for magnetic mRNA isolation can be adapted for use in processing patient samples. In this procedure, the poly(A) tail present in most mRNAs is hybridized in solution to a biotinylated oligo(dT) primer. This is followed by capture using streptavidin-coupled paramagnetic particles and washing at high stringency with SSC. The mRNA is eluted from the SA-PMPs by the addition of ribonuclease-free deionized water. The concentration of the purified mRNA is determined spectrophotometrically. The mRNA is then concentrated by precipitation or vacuum drying for use in applications such as cDNA synthesis or translation in vitro.

It will also be appreciated that a bead-capture system can be adapted to provide the bead as the detection signal. Explained in more detail below, plasmon resonance particles (PRPs) are a means for detecting interaction of a sample analyte and a detection probe. It is contemplated that streptavidin-coupled PRPs or the like can be used in the purification of mRNA from patient samples, then applied to an array of detection probes. Any PRPs which are associated with an mRNA that hybridizes to a detection probe can be detected. In certain embodiments, the PRP is derivatized so that only a few mRNA species can be associated with any single particle, e.g., 1-100 mRNA per particle.

In certain embodiments, it may be desirable to amplify the nucleic acid analytes prior to detection. Nucleic acid used as a template for amplification is isolated from cells contained in the biological sample, according to conventional methodologies. The nucleic acid may be genomic DNA or fractionated or whole cell RNA. Where mRNA is used, it may be desired to convert the mRNA to a complementary cDNA. In one embodiment, the mRNA is whole cell mRNA and is used directly as the template for amplification.

Pairs of primers that selectively hybridize to nucleic acids corresponding to disease state-specific markers are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

Briefly, in PCR, two primer sequences are prepared which are complementary to regions on opposite complementary strands of the marker sequence. An excess of deoxynucleoside triphosphates is added to a reaction mixture along with a DNA polymerase, e.g., Taq polymerase. If the marker sequence is present in a sample, the primers bind to the marker and the polymerase causes the primers to be extended along the marker sequence by adding on nucleotides. By raising and lowering the temperature of the reaction mixture, the extended primers dissociate from the marker to form reaction products, excess primers bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Qbeta Replicase, described in PCT Application No. PCT/US87/00880, may also be used as still another amplification method in the present invention. In this method, a replicative sequence of mRNA which has a region complementary to that of a target is added to a sample in the presence of an mRNA polymerase. The polymerase copies the replicative sequence which may then be detected.

An isothermal amplification method, in which restriction endonucleases and ligases are used to achieve the amplification of target molecules that contain nucleoside 5′-[alpha-thio]-triphosphates in one strand of a restriction site may also be useful in the amplification of nucleic acids in the present invention. Walker et al., (1992) PNAS 89:392-396.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences may also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Other amplification methods are described in GB Application No. 2202328, and in PCT Application No. PCT/US89/01025 may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR. Kwoh et al., (1989) PNAS 86:1173 (1989); Gingeras et al., PCT Application WO 88/10315. In NASBA, the nucleic acids may be prepared for amplification by conventional phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., EPA No. 329 822 disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” Frohman, M. A., In: PCR PROTOCOLS: A GUIDE TO METHODS AND APPLICATIONS, Academic Press, N.Y. (1990) and Ohara et al., Proc. Nat'l Acad. Sci. USA, 86:5673-5677 (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., Genomics 4:560 (1989), incorporated herein by reference in its entirety.

An example of a technique that does not require nucleic acid amplification, that can also be used to quantify mRNA in some applications is a nuclease protection assay. There are many different versions of nuclease protection assays known to those practiced in the art. The characteristic that all versions of nuclease protection assays share in common is that they involve hybridization of an antisense nucleic acid with the RNA to be quantified. The resulting hybrid double stranded molecule is then digested with a nuclease that digests single stranded nucleic acids more efficiently than double stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

In an alternative embodiment, the biological sample obtained from a patient may be first processed to extract fractions containing proteins using any techniques known in the art. For example, the biological sample may be treated with a neutral buffer under conditions of homogenization or other techniques to disrupt the cells and tissues in order to solubilize protein fractions.

II. Determination of Expression Levels of HPE Genes

To generate a test expression profile from a biological sample obtained from a patient, the expression levels of a plurality of genes associated with an oral disease need to be determined. In one embodiment, the plurality of genes used is the set of 45 HPE genes that Applicants have demonstrated to be differentially expressed in oral cancer samples versus normal samples (See Table 1 for a list of the 45 genes).

In an alternative embodiment, a subset of the 45 HPE genes may be used to diagnose an oral disease. A subset of these 45 genes useful for oral cancer diagnosis may be further identified by applying the statistical methods described in the Exemplification section to oral cancer samples and normal samples. The expression profile of the subset of genes may provide a disease signature useful for diagnostic and prognosis purposes. For example, the subset of genes may be less than 20 genes, less than 10 genes, or less than 5 genes. In any case, a subset of genes may be selected, and optionally a corresponding data set of expression levels, rank or concentration, that may be employed to diagnose or detect the indication of interest.

a. mRNA Expression Data

To determine HPE genes and their expression, the invention, in one embodiment, provides a method wherein nucleic acid probes are immobilized on a substrate, such as a microchip, in an organized array (microarray). Oligonucleotides can be bound to a solid support by a variety of processes, including lithography. For example a chip can hold up to 250,000 oligonucleotides (GeneChip, Affymetrix). These nucleic acid probes comprise a nucleotide sequence at least about 12 nucleotides in length, preferably at least about 15 nucleotides, more preferably at least about 25 nucleotides, and most preferably at least about 40 nucleotides, and up to all or nearly all of a sequence which is complementary to a portion of the coding sequence of one or more marker nucleic acid sequence.

Alternatively, the probe immobilized on a substrate can be cDNA, PCR products, proteins, short peptides, or sequences of nucleotide analogs.

The substrate surface of the microarray comprises material selected from the group consisting of polymeric materials, glasses, ceramics, natural fibers, nylon and nitrocellulose membranes, gels, silicons, metals, and composites thereof. Preferably the substrate is glass, more preferably a glass slide. Preferably the microarray substrate comprises at least one flat surface comprising at least one of these materials. Optionally, the substrate is in a form of threads, sheets, films, gels, membranes, beads, plates, and like structures.

To prepare the microarray of the invention, the nucleic acid probes is deposited on the microarray by contacting the nucleic acid probes with an activated substrate by a technique selected from the group consisting of printing, capillary device contact printing, microfluidic channel printing, deposition on a mask, and electrochemical-based printing, wherein the contacting creates a discrete target molecule-containing spot on the substrate (See, for example, U.S. Pat. No. 5,700,637, U.S. Pat. No. 5,445,934, and U.S. Pat. No. 5,807,522 for particular methods of array formation, or Cheung, V. G. et al., Nature Genetics 21(Suppl): 15-19 (1999) for a discussion of array fabrication). It is understood that various additional contacting techniques are well known in the art or may be developed for depositing a target molecule to a solid support. Preferably, a technique is chosen that is accurate, efficient, and economical for the user. The probes can be bound to the substrate by either covalent bonds or by non-specific interactions, such as hydrophobic interactions.

Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734.

A biological sample is applied to the prepared microarray. The biological sample may be collected by employing any suitable sample collection device. The biological sample collected will depend upon the application and may for example be a saliva sample, a tissue swab or scraping, a blood sample, urine sample, or other biological sample. The sample may be preprocessed before being applied to the substrate. In either case, at one point the sample is applied to the microarray to determine the diagnostic or prognostic signatures based on the relative expression of the markers.

In one embodiment, a sample nucleic acid is extracted directly from a sample and contacted with the DNA microarray under conditions sufficient to induce hybridization therebetween, resulting in a hybridization pattern of complementary gene probe/sample complexes. The extracted nucleic acid is preferably RNA, which may be selected from total RNA, poly(A)+RNA, amplified RNA and the like. Methods of isolating RNA from cells, tissues, organs or whole organisms are known to those of ordinary skill in the art and are described, for example, in Maniatis et al., Molecular Cloning: A Laboratory Manual, 2.sup.nd ed., Cold Spring Harbor Laboratory Press, 1989, and in Ausubel et al., Current Protocols in Molecular Biology, John Wiley & Sons, Inc., 1998, the content of each are incorporated herein by reference.

In a further embodiment, sample RNA isolated from a biological sample may be reverse-transcribed to produce cDNA. The cDNA thus produced may be used directly to contact with the DNA microarray. Alternatively, the cDNA may undergo amplifications before contacting with the DNA microarray. The amplification step may be useful in situations where the RNA level for a particular marker gene is low and thus makes detection difficult.

Methods of amplification includes the polymerase chain reaction (PCR), ligation amplification (or ligase chain reaction, LCR), T7 amplification, and amplification methods based on the use of Q-beta replicase. Also useful are strand displacement amplification (SDA), thermophilic SDA, and nucleic acid sequence based amplification (3SR or NASBA). An isothermal amplification method, in which restriction endonucleases and ligases are used to achieve the amplification of target molecules that contain nucleotide 5′-[.alpha.-thio]triphosphates in one strand of a restriction site, may also be useful in the amplification of nucleic acids in the present invention. These methods are well known and widely practiced in the art. See, e.g., U.S. Pat. Nos. 4,683,195 and 4,683,202 and Innis et al., PCR Protocols: A Guide to Methods and Applications (1990) (for PCR); Wu and Wallace, Genomics 4:560-569 (1989) (for LCR); U.S. Pat. No. 6,410,276 and Pabon et al., Biotechniques 31: 874-879 (2001) (for T7 amplification); U.S. Pat. Nos. 5,270,184 and 5,455,166 and Walker et al., Nucl. Acids. Res. 20: 1691-1696 (1992) (for SDA); Spargo et al., Mol. Cell. Probes 10:247-256 (1996) (for thermophilic SDA) and U.S. Pat. No. 5,409,818, Fahy et al., PCR Methods Appl. 1:25-33 (1991) and Compton, Nature 350:91-92 (1991) for 3SR and NASBA, Walker et al., Proc. Natl. Acad. Sci. 89:392-296 (1992) (for isothermal amplification).

In still a further embodiment, the invention involves the additional steps of synthesizing double stranded DNA from messenger RNA in the isolated total cellular RNA, followed by synthesizing RNA complementary (cRNA) to the double stranded DNA. The cRNA is applied to the microarray for measurement.

In another embodiment, the nucleic acid extracted from the biological sample is DNA. This embodiment provides the ability to rapidly analyze the genomic DNA of a marker gene by hybridizing sample DNA with a polynucleotide probe to form a detectable complex. This embodiment is useful in detecting mutations at the DNA level in certain conditions, including Orofacial Clefts, Crouzon Syndrome (Craniofacial Dysostosis), Apert Syndrome (Acrocephalosyndactyly), Treacher Collins Syndrome, and Amelogenesis Imperfecta.

According to one aspect of the invention, the level of expression of target genes can be measured by hybridization with a polynucleotide probe which forms a stable hybrid with that of the target gene sequence, under stringent to moderately stringent hybridization and wash conditions. Double-stranded nucleic acids, comprising the sample nucleic acids bound to probe nucleic acids, can be detected once the unbound portion of the sample is washed away. If it is expected that the probes will be perfectly complementary to the target sequence, stringent conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise. Preferably, the hybridization is performed in a microfluidic system which reduces the necessary reaction time for hybridization and subsequent wash steps.

b. Protein Expression Data

As mentioned above, gene expression level can also be measured at the protein level, by, for example, measuring the levels of polypeptides encoded by the marker gene products. Methods for measuring the levels of polypeptides are well known in the art. For example, immunoassays can be designed based on antibodies to proteins encoded by the nucleic acid sequences. The subject invention provides a method of determining whether a biological sample obtained from a subject possesses an abnormal amount of marker polypeptide which comprises (a) obtaining a sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide.

In an alternate embodiment, the invention provides a method of determining whether a biological sample obtained from a subject possesses an abnormal expression profile of marker genes relative to each other. This method may comprise (a) obtaining a sample from the subject, (b) quantitatively determining the amount of the expression of at least two marker genes relative to each other, and c) comparing the expression profile of marker genes relative to each other so determined with a known standard to determine whether the cell sample obtained from the subject possesses an abnormal expression profile of marker genes relative to each other.

In general, protein expression data may be gathered in any way that, in view of this specification, is available to one of skill in the art. Although many analytical methods provided herein are powerful tools for the analysis of protein data obtained by highly parallel data collection systems, many such methods are equally useful for the analysis of data gathered by more traditional methods.

Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In another embodiment, the invention contemplates using a panel of antibodies which are generated against the HPE polypeptides of this invention, which polypeptides are encoded in Table 1. Such a panel of antibodies may be used as a reliable diagnostic probe for oral cancer.

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissue samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a label for subsequent detection of binding. Samples are incubated for a time sufficient for formation of the immuno-complexes. Binding of the antibody is then detected by virtue of a label conjugated to this antibody. Where the antibody is unlabeled, a second labeled antibody may be employed, e.g., which is specific for the isotype of the anti-marker polypeptide antibody. Examples of labels which may be employed include radionuclides, fluorescers, chemiluminescers, enzymes and the like.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody:enzyme conjugates are readily produced by techniques known to those skilled in the art.

Protein levels may also be detected by a variety of gel based methods. For example, proteins may be resolved by gel electrophoresis, preferably two-dimensional electrophoresis comprising a first dimension based on pI and a second dimension of denaturing PAGE. Proteins resolved by electrophoresis may be labeled beforehand by metabolic labeling, such as with radioactive sulfur, carbon, nitrogen and/or hydrogen labels. If phosphorylation levels are of interest, proteins may be metabolically labeled with a phosphorus isotope. Radioactively labeled proteins may be detected by autoradiography, or by use of a commercially available system such as the PhosphorImager. available from Molecular Dynamics (Amersham). Proteins may also be detected with a variety of stains, including but not limited to, Coomassie Blue, Ponceau S, silver staining, amido black, SYPRO dyes, etc. Proteins may also be excised from gels and subjected to mass spectroscopic analysis for identification. Gel electrophoresis may be preceded by a variety of fractionation steps to generate various subfractionated pools of proteins. Such fractionation steps may include, but are not limited to, ammonium sulfate precipitation, ion exchange chromatography, reverse phase chromatography, hydrophobic interaction chromatography, hydroxylapatite chromatography and any of a variety of affinity chromatography methods.

Proteins expression levels may also be measured through the use of a protein array. For example, one type of protein array comprises an array of antibodies of known specificity to particular proteins. Antibodies may be affixed to a support by, for example the natural interaction of antibodies with supports such as PVDF and nitrocellulose, or, as another example, by interaction with a support that is covalently associated with protein A (see for example U.S. Pat. No. 6,197,599), which binds tightly to the constant region of IgG antibodies. Antibodies may be spotted onto supports using technology similar to that described above for spotting nucleic acid probes onto supports. In another example, an array is prepared by coating a surface with a self-assembling monolayer that generates a matrix of positions where protein capture agents can be bound, and protein capture agents range from antibodies (and variants thereof) to aptamers, phage coat proteins, combinatorially derived RNAs, etc. (U.S. Pat. No. 6,329,209). Proteins bound to such arrays may be detected by a variety of methods known in the art. For example, proteins may be metabolically labeled in the sample with, for example, a radioactive label. Detection may then be accomplished using devices as described above. Proteins may also be labeled after being isolated from the sample, with, for example, a cross-linkable fluorescent agent. In one example, proteins are desorbed from the array by laser and subjected to mass spectroscopy for identification (U.S. Pat. No. 6,225,047). In another variation, the array may be designed for detection by surface plasmon resonance. In this case, binding is detected b changes in the surface plasmon resonance of the support (see, for example, Brockman and Fernandez, American Laboratory (June, 2001) p.37).

III. Detection Techniques

To facilitate detection, the sample nucleic acids or polypeptides extracted from biological sample may be further labeled. The term “label” is used herein in a broad sense to refer to agents that are capable of providing a detectable signal, either directly or through interaction with one or more additional members of a signal producing system. Labels that are directly detectable and may find use in the present invention include, for example, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, and the like; and radioactive isotopes, such as ³⁵S, ³²P, ³H, etc., and the like. In addition, labels may also include near-infrared dyes (Wang et al., Anal. Chem., 72:5907-5917 (2000), upconverting phosphors (Hampl et al., Anal. Biochem., 288:176-187 (2001), DNA dendrimers (Stears et al., Physiol. Genomics 3: 93-99 (2000), quantum dots (Bruchez et al., Science 281:2013-2016 (1998), latex beads (Okana et al., Anal. Biochem. 202:120-125 (1992), selenium particles (Stimpson et al., Proc. Natl. Acad. Sci. 92:6379-6383 (1995), and europium nanoparticles (Harma et al., Clin. Chem. 47:561-568 (2001). The label is one that preferably does not provide a variable signal, but instead provides a constant and reproducible signal over a given period of time.

After labeling, the level of nucleic acids and/or proteins in the sample may be directly detected by a single molecule detection technology. A single molecule detetion technology enables detection at individual molecule level, thus bypassing the need for amplification. The GeneEngine technology coupled with DirectMolecular Analysis developed by U.S. Genomics is an example of direct detection of single biomolecules. Briefly, the molecules are specifically tagged with fluorescence transit through a microfluidic channel under laser spots. Coincident counting rapidly identifies and counts individual target molecules. The data provide information regarding levels of target molecules, molecular identity, intermolecular interactions or other information about individual molecules. See U.S. Pat. Nos. 6,403,311 and 6,355,420, the entire contents of which are incorporated herein.

As an alternative to sample labeling, the system includes direct methods for detecting binding of one or specific binding substrates to their respective analytes that do not require sample labeling. Thus, in one embodiment, the system of the present invention includes a substrate that has a diffractive grating surface. A guided mode resonant phenomenon is used to produce an optical structure that, when illuminated with white light, is designed to reflect only a single wavelength. When molecules are attached to the surface, the reflected wavelength (color) is shifted due to the change of the optical path of light that is coupled into the grating. By linking receptor molecules to the grating surface, complementary binding molecules can be detected without the use of any kind of fluorescent probe or particle label. This techniques is described in more detail in B. Cunningham, P. Li, B. Lin, J. Pepper, “Colorimetric resonant reflection as a direct biochemical assay technique,” Sensors and Actuators B, Volume 81, p. 316-328, Jan. 5, 2002, and in PCT No. WO 02/061429 A2. The spectral shifts may be analyzed to determine the expression data provided, and to indicate the presence or absence of a particular indication.

Accordingly, the system of the present invention may include a biosensor comprising: a two-dimensional grating comprised of a material having a high refractive index, a substrate layer that supports the two-dimensional grating, and one or more detection probes immobilized on the surface of the two-dimensional grating opposite of the substrate layer. When the biosensor is illuminated a resonant grating effect is produced on the reflected radiation spectrum. The depth and period of the two-dimensional grating are less than the wavelength of the resonant grating effect.

A narrow band of optical wavelengths can be reflected from the biosensor when it is illuminated with a broad band of optical wavelengths. The substrate can comprise glass, plastic or epoxy. The two-dimensional grating can comprise a material selected from the group consisting of zinc sulfide, titanium dioxide, tantalum oxide, and silicon nitride.

The substrate and two-dimensional grating can optionally comprise a single unit. The surface of the single unit comprising the two-dimensional grating is coated with a material having a high refractive index, and the one or more detection probes are immobilized on the surface of the material having a high refractive index opposite of the single unit. The single unit can be comprised of a material selected from the group consisting of glass, plastic, and epoxy.

The biosensor can optionally comprise a cover layer on the surface of the two-dimensional grating opposite of the substrate layer. The one or more detection probes are immobilized on the surface of the cover layer opposite of the two-dimensional grating, The cover layer can comprise a material that has a lower refractive index than the high refractive index material of the two-dimensional grating. For example, a cover layer can comprise glass, epoxy, and plastic.

A two-dimensional grating can be comprised of a repeating pattern of shapes selected from the group consisting of lines, squares, circles, ellipses, triangles, trapezoids, sinusoidal waves, ovals, rectangles, and hexagons. The repeating pattern of shapes can be arranged in a linear grid, i.e., a grid of parallel lines, a rectangular grid, or a hexagonal grid. The two-dimensional grating can have a period of about 0.01 microns to about 1 micron and a depth of about 0.01 microns to about 1 micron.

The subject method and systems can make use of any of a variety of biosensor arrays. Biosensors have been developed to detect a variety of biomolecular complexes including oligonucleotides, antibody-antigen interactions, hormone-receptor interactions, and enzyme-substrate interactions. In general, biosensors consist of two components: a highly specific recognition element and a transducer that converts the molecular recognition event into a quantifiable signal. Signal transduction has been accomplished by many methods, including fluorescence, interferometry (Jenison et al., Nature Biotechnology, 19: 62-65; Lin et al., (1997) Science 278:840-843), and gravimetry (Cunningham, Bioanalytical Sensors, John Wiley & Sons (1998)).

Of the optically-based transduction methods, direct methods that do not require labeling of analytes with fluorescent compounds are of interest in certain preferred embodiments due to the relative assay simplicity. Direct optical methods which can be adapted for use in the present invention include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997), plasmom-resonant particles (PRPs) (Schultz et al., Proc. Nat. Acad. Sci., 97: 996-1001 (2000), grating couplers (Morhard et al., “Innnobilization of antibodies in micropattems for cell detection by optical diffraction,” Sensors and Actuators B, 70, p. 232-242, 2000), ellipsometry (Jin et al., “A biosensor concept based on imaging ellipsometry for visualization of biomolecular interactions,” Analytical Biochemistry, 232, p. 69-72, 1995), evanascent wave devices (Huber et al., “Direct optical immunosensing (sensitivity and selectivity),” Sensors and Actuators B, 6, p. 122-126, 1992), resonance light scattering (Bao et al., Anal. Chem., 74:1792-1797 (2002), and reflectometry (Brecht & Gauglitz, “Optical probes and transducers,” Biosensors and Bioelectronics, 10, p. 923-936, 1995).

In general, the subject biosensors include one or more detection probes which can bind to analytes of interest in the processed sample. Exemplary detection probes include nucleic acids (particularly oligonucleotide probes), polypeptides, antigens, antibodies (including polyclonal, monoclonal, single chain antibodies (scFv), F(ab) fragments, F(ab′).sub.2 fragments, Fv fragments, etc), small organic molecules which are ligands for analytes in the processed sample, and the like. To further illustrate, where transcript profiling is the means by which patient samples are evaluated, the subject biosensor can include an array of oligonuleotides which have sequences that hybridize with mRNA or cDNA of interest in establishing a transcript profile for a sample. Molecular arrays useful in the subject methods and kits will preferably include at least 10 distinct detection probes, more preferably at least 25, 50 or 100 different detection probes.

A. Diffractive Grating Surface

In certain embodiments, the subject method utilizes Subwavelength Structured Surface (SWS) or Surface-Relief Volume Diffractive (SRVD) biosensors to study one or a number of detection probe/analyte interactions in parallel. Binding of one or more detection probes to their respective analytes can be detected, without the use of labels, by applying one or more analytes (e.g., in the form of a processed patient sample) to a SWS or SRVD biosensor that have one or more detection probes immobilized on their surfaces.

A SWS biosensor is illuminated with light and a maxima in reflected wavelength, or a minima in transmitted wavelength of light is detected from the biosensor. If one or more detection probes have bound to their respective analytes, then the reflected wavelength of light is shifted as compared to a situation where one or more detection probes have not bound to their respective analytes. Where a SWS biosensor is coated with an array of distinct locations containing the one or more detection probes, such as oligonucleotide probes, then a maxima in reflected wavelength or minima in transmitted wavelength of light is detected from each distinct location of the biosensor.

A SRVD biosensor is illuminated with light after analytes have been added and the reflected wavelength of light is detected from the biosensor. Where one or more detection probes have bound to their respective analytes, the reflected wavelength of light is shifted.

To further illustrate, exemplary biosensors for use in the subject invention can inlcude: a two-dimensional grating comprised of a material having a high refractive index, a substrate layer that supports the two-dimensional grating, and one or more detection probes immobilized on the surface of the two-dimensional grating opposite of the substrate layer. When the biosensor is illuminated a resonant grating effect is produced on the reflected radiation spectrum. The depth and period of the two-dimensional grating are less than the wavelength of the resonant grating effect.

A narrow band of optical wavelengths can be reflected from the biosensor when the biosensor is illuminated with a broad band of optical wavelengths. The substrate can comprise glass, plastic or epoxy. The two-dimensional grating can comprise a material selected from the group consisting of zinc sulfide, titanium dioxide, tantalum oxide, and silicon nitride.

The two-dimensional grating can be comprised of a repeating pattern of shapes, such as lines, squares, circles, ellipses, triangles, trapezoids, sinusoidal waves, ovals, rectangles, and hexagons. The repeating pattern of shapes can be arranged in a linear grid, i.e., a grid of parallel lines, a rectangular grid, or a hexagonal grid. The two-dimensional grating preferably has a period of about 0.01 microns to about 1 micron and a depth of about 0.01 microns to about 1 micron.

(i) Subwavelength Structured Surface (SWS) Biosensor

In one embodiment of the invention, a subwavelength structured surface (SWS) is used to create a sharp optical resonant reflection at a particular wavelength that can be used to track with high sensitivity the interaction of the detection probes with analytes in the processed patient samples. A colormetric resonant diffractive grating surface acts as a surface binding platform for detection probes.

Subwavelength structured surfaces are an unconventional type of diffractive optic that can mimic the effect of thin-film coatings. (Peng & Morris, “Resonant scattering from two-dimensional gratings,” J Opt. Soc. Am. A, Vol. 13, No. 5, p. 993, May; Magnusson, & Wang, “New principle for optical filters,” Appl. Phys. Lett., 61, No. 9, p. 1022, August 1992; Peng & Morris, “Experimental demonstration of resonant anomalies in diffraction from two-dimensional gratings,” Optics Letters, Vol. 21, No. 8, p. 549, April, 1996). A SWS structure contains a surface-relief, two-dimensional grating in which the grating period is small compared to the wavelength of incident light so that no diffractive orders other than the reflected and transmitted zeroth orders are allowed to propagate. A SWS surface narrowband filter can comprise a two-dimensional grating sandwiched between a substrate layer and a cover layer that fills the grating grooves. Optionally, a cover layer is not used. When the effective index of refraction of the grating region is greater than the substrate or the cover layer, a waveguide is created. When a filter is designed properly, incident light passes into the waveguide region and propagates as a leaky mode. A two-dimensional grating structure selectively couples light at a narrow band of wavelengths into the waveguide. The light propagates only a very short distance (on the order of 10-100 micrometers), undergoes scattering, and couples with the forward- and backward-propagating zeroth-order light. This highly sensitive coupling condition can produce a resonant grating effect on the reflected radiation spectrum, resulting in a narrow band of reflected or transmitted wavelengths. The depth and period of the two-dimensional grating are less than the wavelength of the resonant grating effect.

The reflected or transmitted color of this structure can be modulated by the addition of molecules, such as analytes in the sample that bind to the detection probes. The added molecules increase the optical path length of incident radiation through the structure, and thus modify the wavelength at which maximum reflectance or transmittance will occur.

In one embodiment, a biosensor, when illuminated with white light, is designed to reflect only a single wavelength. When detection probes are attached to the surface of the biosensor, the reflected wavelength (color) is shifted due to the change of the optical path of light that is coupled into the grating. By linking detection probes to a biosensor surface, analytes which bind to detection probes can be detected without the use of any kind of fluorescent probe or particle label. Such biosensors can be used with the biosensor surface either immersed in fluid or dried.

An exemplary detection system consists of, for example, a light source that illuminates a small spot of a biosensor at normal incidence through, for example, a fiber optic probe, and a spectrometer that collects the reflected light through, for example, a second fiber optic probe also at normal incidence. Because no physical contact occurs between the excitation/detection system and the biosensor surface, no special coupling prisms are required and the biosensor can be easily adapted to any commonly used assay platform including, for example, microtiter plates and microarray slides. A single spectrometer reading can be performed in several milliseconds, thus it is possible to quickly measure a large number of molecular interactions taking place in parallel upon a biosensor surface.

This technology is useful in applications where large numbers of biomolecular interactions are measured in parallel, such for transcript, antigen or metabolite profiling.

To further illustrate, the two-dimensional grating can be comprised of a material, including, for example, zinc sulfide, titanium dioxide, tantalum oxide, and silicon nitride. A cross-sectional profile of a two-dimensional grating can comprise any periodically repeating function, for example, a “square-wave.” A two-dimensional grating can be comprised of a repeating pattern of shapes selected from the group consisting of lines, squares, circles, ellipses, triangles, trapezoids, sinusoidal waves, ovals, rectangles, and hexagons. A sinusoidal cross-sectional profile is preferable for manufacturing applications that require embossing of a grating shape into a soft material such as plastic. In one embodiment of the invention, the depth of the grating is about 0.01 micron to about 1 micron and the period of the grating is about 0.01 micron to about 1 micron.

Linear gratings have resonant characteristics where the illuminating light polarization is oriented perpendicular to the grating period. However, a hexagonal grid of holes has better polarization symmetry than a rectangular grid of holes. Therefore, a calorimetric resonant reflection biosensor of the invention can comprise, for example, a hexagonal array of holes or a grid of parallel lines. A linear grating has the same pitch (i.e. distance between regions of high and low refractive index), period, layer thicknesses, and material properties as the hexagonal array grating. However, light must be polarized perpendicular to the grating lines in order to be resonantly coupled into the optical structure. Therefore, a polarizing filter oriented with its polarization axis perpendicular to the linear grating must be inserted between the illumination source and the biosensor surface. Because only a small portion of the illuminating light source is correctly polarized, a longer integration time is required to collect an equivalent amount of resonantly reflected light compared to a hexagonal grating.

While a linear grating can require either a higher intensity illumination source or a longer measurement integration time compared to a hexagonal grating, the fabrication requirements for the linear structure are simpler. A hexagonal grating pattern is produced by holographic exposure of photoresist to three mutually interfering laser beams. The three beams are precisely aligned in order to produce a grating pattern that is symmetrical in three directions. A linear grating pattern requires alignment of only two laser beams to produce a holographic exposure in photoresist, and thus has a reduced alignment requirement. A linear grating pattern can also be produced by, for example, direct writing of photoresist with an electron beam. Also, several commercially available sources exist for producing linear grating “master” templates for embossing a grating structure into plastic.

A rectangular grid pattern can be produced in photoresist using an electron beam direct-write exposure system. A single wafer can be illuminated as a linear grating with two sequential exposures with the part rotated 90-degrees between exposures.

A two-dimensional grating can also comprise, for example, a “stepped” profile, in which high refractive index regions of a single, fixed height are embedded within a lower refractive index cover layer. The alternating regions of high and low refractive index provide an optical waveguide parallel to the top surface of the biosensor.

For manufacture, a stepped structure is etched or embossed into a substrate material such as glass or plastic. A uniform thin film of higher refractive index material, such as silicon nitride or zinc sulfide is deposited on this structure. The deposited layer will follow the shape contour of the embossed or etched structure in the substrate, so that the deposited material has a surface relief profile that is identical to the original embossed or etched profile. The structure can be completed by the application of an optional cover layer comprised of a material having a lower refractive index than the higher refractive index material and having a substantially flat upper surface. The covering material can be, for example, glass, epoxy, or plastic.

This structure allows for low cost biosensor manufacturing, because it can be mass produced. A “master” grating can be produced in glass, plastic, or metal using, for example, a three-beam laser holographic patterning process, See e.g., Cowan, (1984) Proc. Soc. Photo-optical Instum. Eng. 503:120. A master grating can be repeatedly used to emboss a plastic substrate. The embossed substrate is subsequently coated with a high refractive index material and optionally, a cover layer.

Techniques for making two-dimensional gratings are disclosed in Wang, (199) J. Opt. Soc. Am 8:1529-44. Biosensors of the invention can be made in, for example, a semiconductor microfabrication facility. Biosensors can also be made on a plastic substrate using continuous embossing and optical coating processes. For this type of manufacturing process, a “master” structure is built in a rigid material such as glass or silicon, and is used to generate “mother” structures in an epoxy or plastic using one of several types of replication procedures. The “mother” structure, in turn, is coated with a thin film of conducive material, and used as a mold to electroplate a thick film of nickel. The nickel “daughter” is released from the plastic “mother” structure. Finally, the nickel “daughter” is bonded to a cylindrical drum, which is used to continuously emboss the surface relief structure into a plastic film. Following embossing, the plastic structure is overcoated with a thin film of high refractive index material, and optionally coated with a planarizing, cover layer polymer, and cut to appropriate size.

A substrate for a SWS biosensor can comprise, for example, glass, plastic or epoxy. Optionally, a substrate and a two-dimensional grating can comprise a single unit. That is, a two dimensional grating and substrate are formed from the same material, for example, glass, plastic, or epoxy. The surface of a single unit comprising the two-dimensional grating is coated with a material having a high refractive index, for example, zinc sulfide, titanium dioxide, tantalum oxide, and silicon nitride. One or more detection probes can be immobilized on the surface of the material having a high refractive index or on an optional cover layer.

A biosensor of the invention can further comprise a cover layer on the surface of a two-dimensional grating opposite of a substrate layer. Where a cover layer is present, the one or more detection probes are immobilized on the surface of the cover layer opposite of the two-dimensional grating. Preferably, a cover layer comprises a material that has a lower refractive index than a material that comprises the two-dimensional grating. A cover layer can be comprised of, for example, glass (including spin-on glass (SOG)), epoxy, or plastic.

For example, various polymers that meet the refractive index requirement of a biosensor can be used for a cover layer. SOG can be used due to its favorable refractive index, ease of handling, and readiness of being activated with detection probes using the wealth of glass surface activation techniques. When the flatness of the biosensor surface is not an issue for a particular system setup, a grating structure of SiN/glass can directly be used as the sensing surface, the activation of which can be done using the same means as on a glass surface.

Resonant reflection can also be obtained without a planarizing cover layer over a two-dimensional grating. For example, a biosensor can contain only a substrate coated with a structured thin film layer of high refractive index material. Without the use of a planarizing cover layer, the surrounding medium (such as air or water) fills the grating. Therefore, detection probes are immobilized to the biosensor on all surfaces a two-dimensional grating exposed to the detection probes, rather than only on an upper surface.

In general, a biosensor of the invention will be illuminated with white light that will contain light of every polarization angle. The orientation of the polarization angle with respect to repeating features in a biosensor grating will determine the resonance wavelength. For example, a “linear grating” biosensor structure consisting of a set of repeating lines and spaces will have two optical polarizations that can generate separate resonant reflections. Light that is polarized perpendicularly to the lines is called “s-polarized,” while light that is polarized parallel to the lines is called “p-polarized.” Both the s and p components of incident light exist simultaneously in an unfiltered illumination beam, and each generates a separate resonant signal. A biosensor structure can generally be designed to optimize the properties of only one polarization (the s-polarization), and the non-optimized polarization is easily removed by a polarizing filter.

In order to remove the polarization dependence, so that every polarization angle generates the same resonant reflection spectra, an alternate biosensor structure can be used that consists of a set of concentric rings. In this structure, the difference between the inside diameter and the outside diameter of each concentric ring is equal to about one-half of a grating period. Each successive ring has an inside diameter that is about one grating period greater than the inside diameter of the previous ring. The concentric ring pattern extends to cover a single sensor location--such as a microarray spot or a microtiter plate well. Each separate microarray spot or microtiter plate well has a separate concentric ring pattern centered within it. All polarization directions of such a structure have the same cross-sectional profile. The concentric ring structure must be illuminated precisely on-center to preserve polarization independence. The grating period of a concentric ring structure is less than the wavelength of the resonantly reflected light. The grating period is about 0.01 micron to about 1 micron. The grating depth is about 0.01 to about 1 micron.

In another embodiment, an array of holes or posts are arranged to closely approximate the concentric circle structure described above without requiring the illumination beam to be centered upon any particular location of the grid. Such an array pattern is automatically generated by the optical interference of three laser beams incident on a surface from three directions at equal angles. In this pattern, the holes (or posts) are centered upon the corners of an array of closely packed hexagons. The holes or posts also occur in the center of each hexagon. Such a hexagonal grid of holes or posts has three polarization directions that “see” the same cross-sectional profile. The hexagonal grid structure, therefore, provides equivalent resonant reflection spectra using light of any polarization angle. Thus, no polarizing filter is required to remove unwanted reflected signal components. The period of the holes or posts can be about 0.01 microns to about 1 micron and the depth or height can be about 0.01 microns to about 1 micron.

The invention provides a resonant reflection structures and transmission filter structures comprising concentric circle gratings and hexagonal grids of holes or posts. For a resonant reflection structure, light output is measured on the same side of the structure as the illuminating light beam. For a transmission filter structure, light output is measured on the opposite side of the structure as the illuminating beam. The reflected and transmitted signals are complementary. That is, if a wavelength is strongly reflected, it is weakly transmitted. Assuming no energy is absorbed in the structure itself, the reflected+transmitted energy at any given wavelength is constant. The resonant reflection structure and transmission filters are designed to give a highly efficient reflection at a specified wavelength. Thus, a reflection filter will “pass” a narrow band of wavelengths, while a transmission filter will “cut” a narrow band of wavelengths from incident light.

A resonant reflection structure or a transmission filter structure can comprise a two-dimensional grating arranged in a pattern of concentric circles. A resonant reflection structure or transmission filter structure can also comprise a hexagonal grid of holes or posts. When these structure are illuminated with an illuminating light beam, a reflected radiation spectrum is produced that is independent of an illumination polarization angle of the illuminating light beam. When these structures are illuminated a resonant grating effect is produced on the reflected radiation spectrum, wherein the depth and period of the two-dimensional grating or hexagonal grid of holes or posts are less than the wavelength of the resonant grating effect. These structures reflect a narrow band of light when the structure is illuminated with a broadband of light.

Resonant reflection structures and transmission filter structures of the invention can be used as biosensors. For example, one or more detection probes can be immobilized on the hexagonal grid of holes or posts or on the two-dimensional grating arranged in concentric circles.

In one embodiment of the invention, a reference resonant signal is provided for more accurate measurement of peak resonant wavelength shifts. The reference resonant signal can cancel out environmental effects, including, for example, temperature. A reference signal can be provided using a resonant reflection superstructure that produces two separate resonant wavelengths. A transparent resonant reflection superstructure can contain two sub-structures. A first sub-structure comprises a first two-dimensional grating with a top and a bottom surface. The top surface of a two-dimensional grating comprises the grating surface. The first two-dimensional grating can comprise one or more detection probes immobilized on its top surface. The top surface of the first two-dimensional grating is in contact with a test sample. An optional substrate layer can be present to support the bottom surface of the first two-dimensional grating. The substrate layer comprises a top and bottom surface. The top surface of the substrate is in contact with, and supports the bottom surface of the first two-dimensional grating.

A second sub-structure comprises a second two-dimensional grating with a top surface and a bottom surface. The second two-dimensional grating is not in contact with a test sample. The second two-dimensional grating can be fabricated onto the bottom surface of the substrate that supports the first two-dimensional grating. Where the second two-dimensional grating is fabricated on the substrate that supports the first two-dimensional grating, the bottom surface of the second two-dimensional grating can be fabricated onto the bottom surface of the substrate. Therefore, the top surface of the second two-dimensional grating will face the opposite direction of the top surface of the first two-dimensional grating.

The top surface of the second two-dimensional grating can also be attached directly to the bottom surface of the first sub-structure. In this embodiment the top surface of the second two-dimensional grating will face the same direction as the top surface of the first two-dimensional grating. A substrate can support the bottom surface of the second two-dimensional grating in this embodiment.

Because the second sub-structure is not in physical contact with the test sample, its peak resonant wavelength is not subject to changes in the optical density of the test media, or deposition of detection probes or analytes on the surface of the first two-dimensional grating. Therefore, such a superstructure produces two resonant signals. Because the location of the peak resonant wavelength in the second sub-structure is fixed, the difference in peak resonant wavelength between the two sub-structures provides a relative means for determining the amount of detection probes or analytes or both deposited on the top surface of the first substructure that is exposed to the test sample.

A biosensor superstructure can be illuminated from its top surface or from its bottom surface, or from both surfaces. The peak resonance reflection wavelength of the first substructure is dependent on the optical density of material in contact with the superstructure surface, while the peak resonance reflection wavelength of the second substructure is independent of the optical density of material in contact with the superstructure surface.

In one embodiment of the invention, a biosensor is illuminated from the bottom surface of the biosensor. Approximately 50% of the incident light is reflected from the bottom surface of biosensor without reaching the active (top) surface of the biosensor. A thin film or physical structure can be included in a biosensor composition that is capable of maximizing the amount of light that is transmitted to the upper surface of the biosensor while minimizing the reflected energy at the resonant wavelength. The anti-reflection thin film or physical structure of the bottom surface of the biosensor can comprise, for example, a single dielectric thin film, a stack of multiple dielectric thin films, or a “motheye” structure that is embossed into the bottom biosensor surface.

In one embodiment of the invention, an interaction of a first molecule with a second test molecule can be detected. A SWS biosensor as described above is used; however, there are no detection probes immobilized on its surface. Therefore, the biosensor comprises a two-dimensional grating, a substrate layer that supports the two-dimensional grating, and optionally, a cover layer. As described above, when the biosensor is illuminated a resonant grating effect is produced on the reflected radiation spectrum, and the depth and period of the two-dimensional grating are less than the wavelength of the resonant grating effect.

To detect an interaction of a first molecule with a second test molecule, a mixture of the first and second molecules is applied to a distinct location on a biosensor. A distinct location can be one spot or well on a biosensor or can be a large area on a biosensor. A mixture of the first molecule with a third control molecule is also applied to a distinct location on a biosensor. The biosensor can be the same biosensor as described above, or can be a second biosensor. If the biosensor is the same biosensor, a second distinct location can be used for the mixture of the first molecule and the third control molecule. Alternatively, the same distinct biosensor location can be used after the first and second molecules are washed from the biosensor. The third control molecule does not interact with the first molecule and is about the same size as the first molecule. A shift in the reflected wavelength of light from the distinct locations of the biosensor or biosensors is measured. If the shift in the reflected wavelength of light from the distinct location having the first molecule and the second test molecule is greater than the shift in the reflected wavelength from the distinct location having the first molecule and the third control molecule, then the first molecule and the second test molecule interact.

a. Detection Probes and Analytes

One or more detection probes are immobilized on the two-dimensional grating or cover layer, if present, by for example, physical adsorption or by chemical binding. A detection probe can be, for example, a nucleic acid, polypeptide, antigen, polyclonal antibody, monoclonal antibody, single chain antibody (scFv), F(ab) fragment, F(ab′)₂ fragment, Fv fragment, small organic molecule, or any other agent which can selectively bind to an analyte of interest in the processed patient sample. A biological sample can be for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

Preferably, one or more detection probes are arranged in a microarray of distinct locations on a biosensor. A microarray of detection probes comprises one or more detection probes on a surface of a biosensor of the invention such that a surface contains many distinct locations, each with a different detection probe or with a different amount of a detection probe. For example, an array can comprise 10, 100, 1,000, 10,000, or 100,000 distinct locations. Such a biosensor surface is called a microarray because one or more detection probes are typically laid out in a regular grid pattern in x-y coordinates. However, a microarray of the invention can comprise one or more detection probe laid out in any type of regular or irregular pattern. For example, distinct locations can define a microarray of spots of one or more detection probes. A microarray spot can be about 50 to about 500 microns in diameter. A microarray spot can also be about 150 to about 200 microns in diameter. One or more detection probes can be bound to their specific analytes.

A microarray on a biosensor for use in the present invention can be created by placing microdroplets of one or more detection probes onto, for example, an x-y grid of locations on a two-dimensional grating or cover layer surface. When the biosensor is exposed to a test sample comprising one or more analytes, the analytes will be preferentially attracted to distinct locations on the microarray that comprise detection probes that have high affinity for the analytes. Some of the distinct locations will gather analytes onto their surface, while other locations will not.

A detection probe specifically binds to a analyte that is added to the surface of a biosensor of the invention. A detection probe specifically binds to its analyte, but does not substantially bind other analytes added to the surface of a biosensor. One example of a microarray of the invention is a nucleic acid microarray, in which each distinct location within the array contains a different nucleic acid molecule. In this embodiment, the spots within the nucleic acid microarray detect complementary chemical binding with an opposing strand of a nucleic acid in a test sample. The biosensor is contacted with the processed patient sample under stringent enough conditions wherein specific hybridization between the detection probe with any complementary nucleic acids in the processed patient sample are highly favored over non-specific hybridization.

b. Immobilization or One or More Detection Probes

Immobilization of one or more binding substances onto a biosensor is performed so that a detection probe will not be washed away by rinsing procedures, and so that its binding to analytes in a test sample is unimpeded by the biosensor surface. Several different types of surface chemistry strategies have been implemented for covalent attachment of detection probes to, for example, glass for use in various types of microarrays and biosensors. These same methods can be readily adapted to a biosensor of the invention. Surface preparation of a biosensor so that it contains the correct functional groups for binding one or more detection probes is an integral part of the biosensor manufacturing process.

One or more detection probes can be attached to a biosensor surface by physical adsorption (i.e., without the use of chemical linkers) or by chemical binding (i.e., with the use of chemical linkers). Chemical binding can generate stronger attachment of detection probes on a biosensor surface and provide defined orientation and conformation of the surface-bound molecules. Examples of chemical cross-linking include, for example, amine activation, aldehyde activation, and nickel activation. These surfaces can be used to attach several different types of chemical linkers to a biosensor surface. While an amine surface can be used to attach several types of linker molecules, an aldehyde surface can be used to bind proteins directly, without an additional linker. A nickel surface can be used to bind molecules that have an incorporated histidine (“his”) tag. Detection of “his-tagged” molecules with a nickel-activated surface is well known in the art (Whitesides, (1996) Anal. Chem. 68:490).

Imobilization of detection probes to plastic, epoxy, or high refractive index material can be performed essentially as described for immobilization to glass. However, the acid wash step can be eliminated where such a treatment would damage the material to which the detection probes are immobilized.

For the detection of analytes at concentrations less than about .about.0.1 ng/ml, it is preferable to amplify and transduce analytes bound to a biosensor into an additional layer on the biosensor surface. The increased mass deposited on the biosensor can be easily detected as a consequence of increased optical path length. By incorporating greater mass onto a biosensor surface, the optical density of analytes on the surface is also increased, thus rendering a greater resonant wavelength shift than would occur without the added mass. The addition of mass can be accomplished, for example, enzymatically, through a “sandwich” assay, or by direct application of mass to the biosensor surface in the form of appropriately conjugated beads or polymers of various size and composition. This principle has been exploited for other types of optical biosensors to demonstrate sensitivity increases over 1500× beyond sensitivity limits achieved without mass amplification. See, e.g., Jenison et al., (2001) Nature Biotechnology 19:62-65.

Merely to further illustrate, a “sandwich” approach can be used to enhance detection sensitivity. In this approach, a large molecular weight molecule can be used to amplify the presence of a low molecular weight molecule. For example, an analyte with a molecular weight of, for example, about 0.1 kDa to about 20 kDa, can be tagged with, for example, succinimidyl-6-[a-methyl-a-(2-pyridyl-dithio) toluamido]hexanoate (SMPT), or dimethylpimelimidate (DMP), histidine, or a biotin molecule. Where the tag is biotin, the biotin molecule will binds strongly with streptavidin, which has a molecular weight of 60 kDa. Because the biotin/streptavidin interaction is highly specific, the streptavidin amplifies the signal that would be produced only by the small analyte by a factor of 60.

Detection sensitivity can be further enhanced through the use of chemically derivatized small particles. “Nanoparticles” made of colloidal gold, various plastics, or glass with diameters of about 3-300 nm can be coated with molecular species that will enable them to covalently bind selectively to a analyte. For example, nanoparticles that are covalently coated with streptavidin can be used to enhance the visibility of biotin-tagged analytes on the biosensor surface. While a streptavidin molecule itself has a molecular weight of 60 kDa, the derivatized bead can have a molecular weight of any size, including, for example, 60 KDa. Binding of a large bead will result in a large change in the optical density upon the biosensor surface, and an easily measurable signal. This method can result in an approximately 1000× enhancement in sensitivity resolution.

(ii). Surface-Relief Volume Diffractive Biosensors

Another embodiment of a biosensor comprises volume surface-relief volume diffractive structures (a SRVD biosensor). SRVD biosensors have a surface that reflect predominantly at a particular narrow band of optical wavelengths when illuminated with a broad band of optical wavelengths. Where detection probes are immobilized on a SRVD biosensor, the reflected wavelength of light is shifted. One-dimensional surfaces, such as thin film interference filters and Bragg reflectors, can select a narrow range of reflected or transmitted wavelengths from a broadband excitation source, however, the deposition of additional material, such as analytes that bind to a detection probe results only in a change in the resonance linewidth, rather than the resonance wavelength. In contrast, SRVD biosensors have the ability to alter the reflected wavelength with the addition of material, such as resulting from the formation of detection probes/analytes complexes.

An SRVD biosensor comprises a sheet material having a first and second surface. The first surface of the sheet material defines relief volume diffraction structures. A sheet material can be comprised of, for example, plastic, glass, semiconductor wafer, or metal film.

A relief volume diffractive structure can be, for example, a two-dimensional grating, as described above, or a three-dimensional surface-relief volume diffractive grating. The depth and period of relief volume diffraction structures are less than the resonance wavelength of light reflected from a biosensor.

A three-dimensional surface-relief volume diffractive grating can be, for example, a three-dimensional phase-quantized terraced surface relief pattern whose groove pattern resembles a stepped pyramid. When such a grating is illuminated by a beam of broadband radiation, light will be coherently reflected from the equally spaced terraces at a wavelength given by twice the step spacing times the index of refraction of the surrounding medium. Light of a given wavelength is resonantly diffracted or reflected from the steps that are a half-wavelength apart, and with a bandwidth that is inversely proportional to the number of steps. The reflected or diffracted color can be controlled by the deposition of a dielectric layer so that a new wavelength is selected, depending on the index of refraction of the coating.

A stepped-phase structure can be produced first in photoresist by coherently exposing a thin photoresist film to three laser beams, as described previously. See e.g., Cowen, (1984) Proc. Soc. Photo-Opt. Instrum. Eng., 503:120-129; Cowen (1985) Opt. Eng. 24:796-802; Cowen et al. (1987) J Imaging Sci. 31:100-107. The nonlinear etching characteristics of photoresist are used to develop the exposed film to create a three-dimensional relief pattern. The photoresist structure is then replicated using standard embossing procedures. For example, a thin silver film is deposited over the photoresist structure to form a conducting layer upon which a thick film of nickel can be electroplated. The nickel “master” plate is then used to emboss directly into a plastic film, such as vinyl, that has been softened by heating or solvent.

The theory describing the design and fabrication of three-dimensional phase-quantized terraced surface relief pattern that resemble stepped pyramids is described: Cowen (1999) J Opt. Soc. Am. A, 7:1529.

An example of a three-dimensional phase-quantized terraced surface relief pattern is a pattern that resembles a stepped pyramid. Each inverted pyramid is approximately 1 micron in diameter, preferably, each inverted pyramid can be about 0.5 to about 5 microns diameter, including for example, about 1 micron. The pyramid structures can be close-packed so that a typical microarray spot with a diameter of 150-200 microns can incorporate several hundred stepped pyramid structures. The relief volume diffraction structures have a period of about 0.1 to about 1 micron and a depth of about 0.1 to about 1 micron. To illustrate how individual microarray locations (with an entire microarray spot incorporating hundreds of pyramids now represented by a single pyramid for one microarray spot) can be optically queried to determine if analytes are adsorbed to detection probes on the surface, when the structure is illuminated with white light, structures without significant bound material will reflect wavelengths determined by the step height of the structure. When higher refractive index material, such as resulting from complexes formed by analytes and detection probes, are incorporated over the reflective metal surface, the reflected wavelength is modified to shift toward longer wavelengths. The color that is reflected from the terraced step structure is theoretically given as twice the step height times the index of refraction of a reflective material that is coated onto the first surface of a sheet material of a SRVD biosensor. A reflective material can be, for example silver, aluminum, or gold.

One or more detection probes, as described above, are immobilized on the reflective material of a SRVD biosensor. One or more detection probes can be arranged in microarray of distinct locations, as described above, on the reflective material. For example, many individual grating structures, represented by small circles, can lie within each microarray spot. The microarray spots, represented by the larger circles, will reflect white light in air at a wavelength that is determined by the refractive index of material on their surface. Microarray locations with additional adsorbed material will have reflected wavelengths that are shifted toward longer wavelengths, represented by the larger circles.

Because the reflected wavelength of light from a SRVD biosensor is confined to a narrow bandwidth, very small changes in the optical characteristics of the surface manifest themselves in easily observed changes in reflected wavelength spectra. The narrow reflection bandwidth provides a surface adsorption sensitivity advantage compared to reflectance spectrometry on a flat surface.

An SRVD biosensor reflects light predominantly at a first single optical wavelength when illuminated with a broad band of optical wavelengths, and reflects light at a second single optical wavelength when one or more detection probes are immobilized on the reflective surface. The reflection at the second optical wavelength results from optical interference. A SRVD biosensor also reflects light at a third single optical wavelength when the one or more complexes are formed from immobilized detection probes and their respective analytes, due to optical interference.

Readout of the reflected color can be performed serially by focusing a microscope objective onto individual microarray spots and reading the reflected spectrum, or in parallel by, for example, projecting the reflected image of the microarray onto a high resolution color CCD camera.

An SRVD biosensor can be manufactured by, for example, producing a metal master plate, and stamping a relief volume diffractive structure into, for example, a plastic material like vinyl. After stamping, the surface is made reflective by blanket deposition of, for example, a thin metal film such as gold, silver, or aluminum. Compared to MEMS-based biosensors that rely upon photolithography, etching, and wafer bonding procedures, the manufacture of a SRVD biosensor is very inexpensive.

B. Plasmon Resonant Entities (PREs)

Yet another embodiment, the detection of mRNA transcripts or other analytes in the processed patient sample utilizes surface plasmon resonant particles (PRPs), also called resonance light scattering particles (RLSs), for signal amplification, e.g., to avoid or at least reduce the need for amplication of the analyte. In general, PRP particles are particles of silver or gold (other materials) that scatter light. The size and shape of PRP particles determines the wavelength of scattered light. See, for example, Schultz et al., (2000) PNAS 97:996-1001; and Yguerabide et al. (1998) Anal Biochem 262: 137.

“Plasmon resonant particle” or “PRP” denotes a single piece or fragment of material, e.g., spherical particle, which elicits plasmon resonance when excited with electromagnetic energy. A plasmon resonant particle can be “optically observable” when it exhibits significant scattering intensity in the optical region, which includes wavelengths from approximately 180 nanometers (nm) to several microns. A plasmon resonant particle can be “visually observable” when it exhibits significant scattering intensity in the wavelength band from approximately 400 nm to 700 nm which is detectable by the human eye. Plasmon resonance is created via the interaction of incident light with basically free conduction electrons. The particles or entities have dimensions, e.g., diameters preferably about 25 to 150 nm, more preferably, about 40 to 100 nm.

The term “plasmon resonant entity” or “PRE” is used herein to refer to any independent structure exhibiting plasmon resonance characteristic of the structure, including (but not limited to) both plasmon resonant particles (PRPs) and combinations or associations of plasmon resonant particles as defined and described above. A PRE may include either a single PRP or an aggregate of two or more PRPs which manifest a plasmon resonance characteristic when excited with electromagnetic energy.

A “field having a plurality of PREs distributed therein” is a one-, two-, or three-dimensional region, for example, a microarray or portion or region of an array having PREs attached or otherwise distributed therein, such that the PREs in the field, when illuminated with an optical light source, exhibit plasmon resonance.

A “spectral emission characteristic” is a term that encompasses a spectral scattering characteristic of a PRE related to the plasmon resonance of the PRE. As used herein, “emission”, as applied to PREs, means scattered light produced or excited by plasmon resonance.

The “value” of a spectral emission characteristic is the qualitative or quantitative value of the emission feature, e.g., the value of the detected peak intensity, peak wavelength, or peak width at half maximum.

A “selected spectral signature” is a term that encompasses a selected range of values of a selected spectral emission characteristic, e.g., a range of spectral peak intensity values.

A “computer image of the positions and values of the emission spectral characteristic” is a term that encompasses to a matrix which associates each region in a field being interrogated with one or more spectral emission characteristic values or signature measured for a light-scattering entity in that region. The image may be a matrix of stored values, or may be an actual image showing the locations of light-scattering entities in one dimension or plane, e.g., the x-y plane, and the associated spectral emission value in another dimension, e.g., the z-axis.

Plasmon resonant entities (PREs) or plasmon resonant particles (PRPs) scatter incident light, and the resulting scattered light has a frequency spectrum characteristic of the particle. A general theory describing the interaction of an incident electromagnetic wave with a spherical particle which successfully predicts this resonant scattering was developed early in the 20th century (H. C. Van Ve Hulst, Light Scattering by Small Particles, Wyley, N.Y., 1957). In a metallic sphere, the incident electromagnetic field induces oscillations, referred to as “plasmons”, in the nearly free conduction electrons of the metal, and these plasmons produce an emitted electromagnetic field. For some materials, and for the optimum choice of particle size, shape, and morphology, there will be a maximum scattering efficiency at a wavelength characteristic of the scattering particle and its surrounding medium. For some materials, the intensity of the emitted light is sufficient for observation under an optical microscope. Silver particles are the most notable exhibitors of this effect, as the wavelength of the resonantly scattered light can be in the visible region of the spectrum.

Theoretical calculations correctly predict that the resonantly scattered wavelength of a spherical particle will increase, or be “red-shifted”, with increasing particle diameter and with increasing dielectric constant of the surrounding material. For spherical particles, dipole resonance produces a scattered frequency spectrum having a single peak at a wavelength which is dependent on the material the particle is made from the size of the particle, the shape of the particle, the morphology of the particle, and the local environment. Larger particles have a longer dipole scattering peak wavelength, and smaller particles have a shorter dipole scattering peak wavelength. The spectrum of scattered light may also contain contributions from a particle's quadrupole resonance. For a given shape, a resonant particle scatters predominantly in a particular wavelength band depending on the composition and size of the particle.

The conductive portion responsible for the plasmons can take many different forms, including solid geometric shapes such as spheres, triangular parallelpipeds, ellipsoids, tetrahedrons, and the like, or may comprise spherical, cylindrical, or other shape shells. It is also true that a dielectric sphere of similar dimensions, having silver or gold on its surface will also exhibit plasmon resonances, assuming the shell has a thickness of at least about 3 nm, preferably 5 nm or more.

It can further be appreciated that contact or near contact between two plasmon resonant particles will produce an electromagnetic coupling between the particles, thereby producing an entity with properties in some ways similar to a single particle having a size equal to the sum of the two particles in contact. Aggregations of many plasmon resonant particles can therefore also exhibit plasmon resonance with characteristics dependent on the geometry and nature of the conglomerate.

Another feature of plasmon creation in a metallic particle is the generation of enhanced electric fields in the region near its surface. Interactions between this electric field and nearby materials can significantly alter both the scattering characteristics of the resonant particle and the nearby material. For example, Surface Enhanced Raman Spectroscopy (SERS) exploits the localized plasmon resonance in roughened or particle coated silver films to enhance the Raman scattering of various materials by as much as six orders of magnitude. In this technique, Raman scattering from the materials of interest is observed, and the local field generated by the plasmons is used to enhance the intensity of that scattering.

In one embodiment, an array of detection probes are provided on a surface, such as a glass, silicon or plastic chip. Processing of patient samples includes treating the analytes, e.g., mRNA or cDNA, with PREs that can be associated by covalent or non-covalent interactions—but in a manner which does not interfere with binding of the analyte to its cognate detection probes. Simply to illustrate, oligo-dT linked PREs can be contacted with mRNA from a processed patient sample in order to produce complexes of PRE/mRNA linked by basepair hybridization between the poly-A tail of the mRNA and the oligo-dT of the PRE. The complexes can then be applied to a microarray of relevant nucleic probes, and detection of the hybridization of the coding sequence of the mRNA to a probe on the array is enhanced by the fact that the PRE is associate with the mRNA.

To further illustrate, different PREs can have differences in spectral characteristics that are easily detected, which permits for use of PREs in embodiments where the detection probes for different analytes are not located in discrete spots, but rather are overlapping or even complete coincident in their location on a substrate.

(i) Method and Apparatus for Interrogating a Field

In one aspect, the subject method and apparatus are designed for interrogating a field which may have a plurality of PREs distributed therein. In such embodiments, the method has three parts, in essence: (i) generating data about one or more spectral emission characteristic(s) of PREs in the field, (ii) from this data, constructing a computer image of the PRE positions (regions in a field) and values of the emission spectral characteristic of individual PREs and other light-scattering entities present in the field, and (iii) by discriminating PREs with selected spectral characteristics in the image from other light-scattering particles in the field, providing information about the field, e.g., a target in the field.

a. Spectral Emission Characteristics

The invention contemplates detecting one or more of several types of spectral emission characteristics, for generating an image of light-scattering particles in the field. The spectral emission characteristics of interest may be plasmon-resonance spectral features of a single PRP, a shift in spectral emission feature due to the interaction of two or more PRPs in close proximity, or a fluorescent or Raman spectroscopic feature induced by the enhanced local electric fields interacting with fluorescent, luminescent, or Raman molecules localized on PREs. The most important of characteristics, and the type of information available from each, are the following.

Peak wavelength is the wavelength of the peak of the spectral emission curve, that is, the wavelength at which maximum intensity occurs. The peak wavelength value can be determined in one a number of different ways, seven of which are described here. The implementation of each of the methods will be understood from the disclosed method, and for some of the methods, as discussed below in the description of the light source and detector in the apparatus of the invention. All of these methods are applicable to measuring the spectral curves for a plurality simultaneously. It will be appreciated that some of the methods are also applicable to measuring the spectral curve of each light-scattering entity in the field individually, for example, by rastering a photodetector element over the plane of the field.

(i) The field is illuminated over a range of illuminating wavelengths, for example, at each of a series of narrowband illumination windows through the visible light spectrum. Typically, a filter wheel interposed between a white light source and the field is employed to generate the narrowband illumination frequencies.

(ii) Light emitted from the field is directed through a dispersive element, such as a prism, for breaking the emitted light into several narrowband frequencies, which are then each directed to a separate detector array. As an example, a prism is used to break the emitted light into red, green and blue components, each directed onto a separate CCD array.

(iii) Take the emitted field image into a dense bundle of optical fibers, through a lens that, for example, magnifies each light-scattering spot corresponding to a PRE, such that its image fits entirely in the core diameter of an optical fiber. Each fiber is then broken up by a dispersion element into spread out spectrum line of different frequencies, which is then read by a line of detector elements in a two dimensional array. Thus each line in the field is read by a 2-dimensional array, one array dimension corresponding to the spectral intensity at each of a plurality of frequencies, and the other dimension, to different positions along an axis in the field. This approach allows for simultaneous reading of a plurality of PREs at each of a plurality of spectral wavelengths.

(iv) Illuminate with multiple narrow band light sources, e.g., 3 or 4 separate laser lines in the red, green, yellow and blue. Each laser is chopped at a different frequency, typically all under 100 Hz. The emitted light from the field is detected in a CCD that can be read at 100 frames/sec.

Computer analysis involving standard techniques is then used to determine the amount of light of each color impinging on each pixel in the CCD array, thereby allowing the spectral emission curve to be constructed.

(v) The same information may be obtained by routing the scattered light through an interferometer, as described for example, in U.S. Pat. No. 5,539,517.

(vi) It is also a property of plasmon resonant particles that the scattered light undergoes a 180 degree phase shift relative to the incident light as the wavelength of incident light is swept through the resonant peak. At the peak wavelength, the phase difference is 90 degrees. This phase shift can be detected, and the peak scattering wavelength can be determined as that incident wavelength when a phase shift of 90 degrees is observed.

(vii) The intensity of PRE light emission at a plurality of defined bandwidths can also be determined by exposing the PREs to short pulses of incident light of varying duration. In particular, it is effective to use pulses approximating a step function increase or decrease, that is, with fast rise time or decay time of only 1 or 2 femtoseconds. The scattering response of a PRE is that of a forced and damped oscillator, and near the resonant wavelength, the response of a PRE to narrowband excitation increases as the excitation pulse length increases. Away from the resonant wavelength, the response to narrowband excitation is small, and relatively independent of the excitation pulse length. Exposing a PRE to pulses of varying duration, but all advantageously less than about 500 femtoseconds, at a particular wavelength and noting how long it takes for the emitted energy to reach a steady state value provides information about how close that particular wavelength is to the PRE resonant wavelength. By exciting the PREs to several series of duration variable pulses, wherein each series has a different peak wavelength, a curve of scattering cross section versus wavelength can be generated.

The peak wavelength generally shifts toward the red (longer wavelengths) as the size of the PRE increases for silver and gold PREs. Peak wavelength values can also provided information about PRE shape. Shape changes from spherical to hexagonal or triangular result predominantly a shift of peak wavelength toward the red. Dielectric-shell PRPs, i.e., particles composed of an inner dielectric core encased in a conductive metal also tend to have longer peak wavelengths than solid metal particles of the same size.

Peak intensity is the intensity of the peak of the spectral emission curve, and may be expressed as an absolute or relative intensity value. The peak intensity value is determined, as above, by one of a variety of methods for determining the spectral emission curves of the PREs, with intensity being determined at the peak wavelength. The peak intensity will vary with material, morphology and shape. For a particular PRE, the intensity will be a maximum in the pane of focus.

Width at half peak height is the width, in wavelength units, of the spectral emission curve at half peak intensity. This value may be measured as an independent spectral characteristic, or combined with peak spectral intensity to characterize the spectral emission curve, for example, the ratio of peak intensity/peak width. Generally peak width increases with increasing size of the PRE, and changes as the shape of the PRE changes from spherical to non-spherical shapes in a manner which can be simulated.

Width in the image plane is the halfwidth of the central diffraction region in the Airy pattern in the image plane. All PRPs are sub-wavelength sources of light, and so their spatial image will be an approximate point spread function with characteristics defined by the optical system being used. Assuming that the optical system includes a CCD, with a pixel array of photodetecting elements, the width of the central diffraction region, which may cover several pixels, is measured radially from the peak of the center of the diffraction image to the position in the center of the image where the intensity has fallen to half its peak value (assuming a circular image).

Since the PRPs are subwavelength scatterers, the halfwidth of the intensity pattern as recorded in the image plane will be proportional to the wavelength of light being scattered. Therefore, for a reasonably smooth variation in light intensity from a source (such as a Xenon arc), the light is scattered most strongly is at peak intensity, and one can make a good estimate of peak wavelength by measuring the width of the half intensity of the central diffraction region in the image for each PRP.

As will be seen below, this spectral characteristic is useful for precise determination of the positions of PREs in a field, and particularly for determining the distance between two PREs of different peak wavelengths that are more closely spaced than the Rayleigh resolution distance. The intensity of the peak of the diffraction pattern in the image plane can be used for focusing the detector lens on the field, with the maximal value giving the best focus.

Polarization measures a spectral characteristic, e.g., peak wavelength, peak height, width at half wavelength, or width at half peak intensity in the image plane, as a function of direction of polarization of light illuminating a PRE field, or the angle of incidence of polarized light. The polarization characteristic depends on PRE shape rather than size, and is due to the fact that a non-spherical PRE may have more than one resonance, for example, along the directions of the major and minor axes in an elliptical PRE. In the latter case, illuminating light directed along the major axis would be shifted toward the red, while that directed along the minor axis, would be shifted toward the blue.

Pulse or time response provides a measure of the number of light cycles of the illuminating light that are required to “pump up” the scattering to full intensity. PREs have very fast time response (sub-picosecond), and very large pulses of scattered photons can be generated, the only limitation being the average input power absorbed. They can accept pulses between 5 to 500 femtosecond for driving two-photon processes or second harmonic generation and other higher order processes.

As noted above, pulsed or timed illumination measurements are generally made by exposing PREs in the field to short pulses of incident light of varying duration, to detect peak wavelength. The time to full resonance, as measured by intensity versus pulse time, also provides a measure of the quality of the material as a plasmon resonator. Higher quality material is characterized by a narrower width of the resonance signature, a higher peak intensity, and a longer time to reach the maximum intensity of scattering when illuminated by pulses of light at the peak wavelength.

Phase shift is discussed above in the context of determining spectral peak at 90 degree phase shift. Phase shift can also give information about the response for excitation wavelength away from the resonant peak wavelength.

Fluorescence emission lifetime can be observed in PRE particles having surface-localized fluorescent molecules. The fluorescence excitation can be enhanced by the local electric fields generated near the surface of the PRE by light within the plasmon resonance peak. Fluorescence emission can also be enhanced if the wavelength of the fluorescence emitted light is within the plasmon resonance peak. Under appropriate conditions, the fluorescence lifetime can be measurably shortened in this process.

The method can be used to detect changes in the excitation environment of the fluorescent molecules, e.g., proximate interactions with other molecules or entities.

Surface enhanced Raman scattering (SERS) relies on the generation of enhanced electric fields in the region near the surface of a PRE. Interactions between this electric field and nearby materials can significantly alter both the scattering characteristics of the resonant particle and the nearby material. Surface Enhanced Raman Spectroscopy (SERS) traditionally exploits the localized plasmon resonance in roughened or particle evaporated silver films to enhance the Raman scattering of various materials by as much as six orders of magnitude. The SERS performed in accordance with the present invention is confined solely to PREs. In this technique, Raman scattering from the materials of interest is observed, and the local field generated by the plasmons is used to enhance the intensity of that scattering by many orders of magnitude over traditional SERS. When the Raman active molecule has a resonant absorption near peak of the spectral emission curve of the PRE, the additional SERS enhancement is sufficient to make the Raman signal of the PRE-molecule composite detectable, in accordance with the method of the invention disclosed in Section III. Measuring changes in the PRE resonant Raman spectrum can be used to detect alterations, e.g., binding, in the local environment of the Raman molecule.

b. Field To Be Interrogated

To continue the illustration, the field that can be interrogated, in accordance with the method and apparatus of the invention, includes a region of a microarray having a plurality, i.e., two or more detection probes distributed in the field.

Methods for forming PREs and preparing a field having PREs distributed therein will be discussed in detail below. At this point, three general cases will be briefly considered. First, preformed PREs-associated analytes are added to an array of detection probes. The array may be washed to remove unbound or non-specifically bound PREs. Second, nucleation sites may be added to the target. After binding to selected locations on the array, a metal enhancer solution, e.g., silver solution, is added until an appropriately sized PRE is formed. In the third case, PREs are formed by photolithographic methods, e.g., photomasking and photoetching, on a metal substrate, e.g., silver substrate.

The types of information which one wishes to determine, by interrogating the field containing the detection probes and any associated PRE/analytess, in accordance with the invention include: (i) the total number of PREs of a selected type in the field, (ii) the spatial pattern of PREs having a selected spectral characteristic in the field, (iii) a distance measurement between two adjacent PREs, particularly PREs separated by a distance less than the Rayleigh resolution distance, (iv) a change in the environment of the field, e.g., dielectric constant, that affects a plasmon resonance characteristics, (v) whether two PREs are linked, or (vi) a fluorescence or Raman emission of molecules or materials attached localized on PREs. Other types of information, are also contemplated.

c. Apparatus

In an exemplary embodiment, an array to be interrogated is supported on a substrate held on a microscope stage which is selectively movable in the x-y plane under the control of a stage stepper-motor device, such as under the control of a computer, which may also include other computational components of the apparatus as described below.

The target is illuminated by an optical light source which directs illuminating light, typically light in the visible range, and at one or more selected wavelength ranges, onto the array surface. As will be detailed below, the light source typically includes a means for generating light of a given wavelength or spectral frequency, one or more filters for producing a desired frequency band of illuminating light, and a lens system for focusing the light onto the target.

Spectral emission light from the target, in this case light scattered from the target, is directed through lens to an optical detector. The optical detector functions to detect one or more spectral emission characteristics of the individual PREs in the illuminated portion of the field. The detector can be, for example, a CCD (Charge Coupled Device) array which operates to generate and store an array of optical intensity values corresponding to the array pixels.

An image processor contained within computer is operatively connected to the detector to receive values of light intensity at each of the detector array positions, under each selected illumination condition, e.g., different wavelength or polarization state. The image processor functions to construct a computer image of the positions and values of one or more spectral emission characteristics measured by the detector. Typically, this is done by treating each pixel in the detector array as a position point in the illuminated field, and assigning to each pixel “position” the light intensity value recorded by that pixel. The image generated by the image processor may be a matrix of stored numbers, e.g., position coordinates and associated spectral emission characteristic value(s), or an actual map in which position are represented, for example, in an x-y plane, and each measured spectral emission value, represented as a quantity along the z axis, for each pixel location.

A discriminator in the apparatus, also forming part of computer, functions to discriminate PREs with a selected spectral signature, i.e., a selected range of values of one or more selected spectral emission characteristics, from other light-scattering entities in the computer image.

c1. Substrate

As indicated above, the detection probes are supported on a substrate which is mounted on a microscope stage. Suitable substrates include standard glass slides, cover slips, clear polystyrene, and clear mica as examples. Other suitable transparent substrates are those associated with a TEM grid, including for example, formvar, carbon and silicon nitride. These TEM-associated substrates are all optically transparent at the thicknesses used. Conducting, semiconducting, and reflecting substrates are also suitable for PRE applications.

Another suitable substrate for use in the present invention are those which may initially appear opaque to the spectral wavelengths of interest for PRE observation, but which can be rendered suitable by the application of a suitable fluid or vapor. An example is white nitrocellulose “paper” as used for the transference of biological samples of interest in diagnostic techniques such as “Southerns”, “Northerns”, “Westerns”, and other blotting, spotting, or “dip stick” tests. Once the materials of interest have been transferred and fixed as desired, the PRE's can be applied as preformed entities, or one can apply PRE nucleation entities and enhance as described below. The white nitrocellulose at this stage may typically present significant non-specular light scattering which makes it difficult to visualize the PREs. However, if a suitable treatment which results in a significant reduction of the non-specular scattering is used, for example, allowing acetone vapor to encompass the nitrocellulose substrate, while monitoring the PREs, the substrate can become much less opaque, and permit efficient observation of the PREs.

Silicon is a preferred substrate for many PRE detection applications because it can be made very smooth and free of defects, resulting in very little non-specular scattering under darkfield illumination. One example of a particularly preferred silicon substrate is the highly polished, etched, and defect free surfaces of silicon wafers commonly used in the manufacture of semiconductors. The nearly complete absence of contaminants and surface imperfections of such a substrate produces excellent contrast of the PRE scattering under darkfield illumination conditions. However, it should be appreciated that such silicon wafers typically have a thin layer of SiO₂ present on their surface as a result of the various processing steps. In other systems, silicon substrates with approximately 100 nm or more of SiO₂ on their surface produce some of the most intense, high contrast PRE spectra so far observed from a solid substrate, and it may be advantageous to intentionally grow a sub-micron layer of SiO₂ on the silicon wafer surface.

If the oxide layer is removed from the silicon surface in a manner that prevents rapid re-growth of an oxide layer, for example, by etching in HF acid and passivating the surface with hydrogen, the optical image of the “point-source” PREs has been observed to be torus-shaped, rather than the usual Airy ring pattern with a bright central region. This “doughnut” phenomenon most likely arises as a result of damping of the transverse driving electric fields (those parallel to the silicon surface), leaving only the perpendicular driving fields which can excite a plasmon mode that radiates well, but not at all directly along the normal. This property of bare silicon substrates can be useful in determining whether a particular PRE is closely bound to the surface of the silicon substrate, or is bound via a tether molecule or system that has placed it further from the surface, thereby changing the dipole component scattering ratios.

c2. Light Source and Detector

Continuing with the exemplary embodiment, light-generating means, e.g., a light source, may suitably be a mercury, xenon, or equivalent arc; or a Quartz-tungsten halogen bulb, of approximately 20 to 250 watts, which provides incident light in a frequency band corresponding to wavelengths from approximately 350 nm to 800 nm, for visible light PRE scattering, or a conventional UV source for lower-wavelength PRE scattering.

The filters typically include a set of pre-selected narrow bandwidth filters, allowing manual or computer controlled insertion of the respective filters. The bandwidth for such filters is typically 5-10 nm.

Other methods of illuminating a target with a series of selected bandwidths include the use of light sources such as lasers of all types where one may utilize very narrow bandwidths. Multiple frequency sources are also contemplated, such as tuned lasers (i.e. Ar-ion) to select any of the characteristic defined strong “line” sources. Alternatively a grating or prism monochrometer can be used. All the light sources can be either of continuous or pulsed variety, or a suitable light amplitude modulation device can be inserted in the incident path to vary the intensity level in a prescribed temporal manner. The polarization of the light to be incident upon the sample can be varied by the insertion of suitable filters or other devices well known to the art.

The microscope can be be configured with an epi-illumination system, whereby the collimated light from the source following filtering as desired impinges onto a half silvered mirror, and is reflected downwards towards the Darkfield/Brightfield (DF/BF) lens. In this particular type of DF/BF application, the incident light that would have had rays passing through the objective lens is physically blocked by an opaque circle, which is suspended by very fine webs, so as to allow only a concentric band of light to pass. The unit comprising one or more mirrors and opaque circle may be built into an adjustable block that can be manually (or robotically) moved thereby converting the microscope from DF/BF to alternate forms of operation.

Light reflected from the mirror may in turn be refracted or reflected (by a suitable circular lens element, fixed to the objective lens mount into a hollow cone of incident light, converging toward a focus at the sample plane of the target. As previously noted, the specular reflection of such rays causes them to return along the lines of the incident cone trajectories, where they are ultimately absorbed or otherwise removed from the optical system.

In this exemplary darkfield system, the angle between the optic axis and the incident rays illuminating the sample is larger than the largest angle between the optic axis and the rays scattered by the PREs which is accepted into the objective lens element, which is illustrated to be of the refractive form. Also incorporated in the total optical microscope is the ability to divert the light rays away from detector to other ports whereby the image may be observed visually through standard binocular eyepieces, or to yet another port, for example, for photographing the illuminated field.

Various image capture devices known in the art may be used, including fiber coupled photo-diode arrays, photographic film, etc. One exemplary device is a thermoelectrically cooled CCD array camera system, model CH250, manufactured by Photometrics, of Tucson Ariz. This device utilizes a CCD chip model KAF1400, having a 1032 by 1037 pixel array.

It will be appreciated that the detector serves to detect a spectral emission characteristic of individual PREs and other light-scattering entities in the field, when the field is illuminated by the light source, simultaneously at each of the regions in the field corresponding to array pixels.

c3. Image Processing Discrimination and Output

Where the detector is used, for example, to detect spectral peak wavelength, peak intensity, and/or half width of the spectral peak, the detector measures light intensity at each of a plurality of different illuminating light frequencies, simultaneously for each of the field regions corresponding to a detector array pixel.

The emission (scattering) values measured at each frequency are stored, allowing spectral emission curves for each region to be constructed after a full spectrum of illumination. From these curves, peak wavelength, peak intensity, and width at half intensity are calculated for each region. Similarly, the peak halfwidth in the image plane can be measured with a CCD array as described above.

The detector may be supplied with comprehensive software and hardware that allows timed exposures, reading out of the pixels into suitable files for data storage, statistical analysis, and image processing (as one of the functions of computer). This capability serves as an image processor for constructing from signals received from the detector, first the values of the spectral emission characteristic(s) being determined, and then a computer image of these values and the corresponding associated field positions.

The image constructed by the image processor may be a matrix of stored points, e.g., a matrix of associated values of each field position (regions in the field) and values for one or more measured spectral characteristics, or may be an actual map of field positions, e.g., in the x-y plane, and associated spectral emission values in the z plane.

The computer in the apparatus also provides discriminator means for discriminating PREs with a selected spectral signature from other light-scattering entities in the computer image. The basis for this discrimination is noted above in the discussion of various spectral emission characteristics and their correlation with physical properties of light-scattering entities.

Thus, for example, to discriminate PREs with a selected spectral peak wavelength and peak width at half intensity, the computer image generated could provide a matrix of all field regions and the associated spectral peak wavelength and width values. The discriminator would then selected those regions containing PREs whose spectral signature meets certain ranges of these two spectral emission values. Depending on the particular values chosen, the discriminator could classify light-scattering entities in the field in a number of ways, including distinguishing:

1. PREs with a selected spectral signature from all other light-scattering entities in the field;

2. PREs from non-PRE light scattering entities in the field;

3. For a selected type of PREs, those selected PREs which are interacting with one another and those which are not; and

4. One selected type of PRE from another selected type of PRE in the field.

In each case, the basis for the discrimination may be based on detected values, for each light-scattering entity in the field, of peak position, peak intensity, or peak width at half intensity of the spectral emission curve, peak halfwidth in the image plane, and polarization or angle of incidence response. Other spectral characteristics mentioned above are also contemplated. In particular, where the PREs have surface-localized fluorescent molecules or Raman-active molecular entities, the detecting may detecting plasmon-resonance induced fluorescent emission or Raman spectroscopy emission from one or more of said molecules or entities, respectively, and these values are used as a basis of discriminating such PREs from other light-scattering entities.

The information obtained from the discriminating step is then used to provide information about the field. Among these are:

1. The total number of PREs of a selected type in a field. Here the discriminating step includes counting the number of PREs having a selected range of values of a selected spectral emission characteristic in the constructed computer image;

2. Determining a spatial pattern of PREs having a selected range of values of a selected spectral characteristic in the field. Here the discriminating includes constructing an image of the relative locations of PREs with those spectral-characteristic values;

3. The distance between two adjacent PREs, particularly where this distance is less than the Rayleigh resolution distance. Here the detecting includes exposing the field with light of one wavelength, to obtain a diffraction image of PREs in the field, exposing the field with light of a second wavelength to obtain a second diffraction image of PREs in the field, and comparing the distance between peaks in the two diffraction patterns;

4. Interrogating a change in the environment of the field. Here the discriminating includes comparing the values of the detected spectral characteristic of a PRE in the field before and after the change, e.g., change in the dielectric of the field;

5. Detecting motion of PREs in the field. The detecting here includes detecting the centers of the diffraction patterns of the PREs in the image plane, as a function of time.

C. Branched Hybridization

Yet another detection technique which can be adapted for use in the present invention is branched-DNA (bDNA) signal-amplification technology. This technology has been used extensively in a microwell format to detect and quantify specific nucleic acid sequences, particularly as it has the sensitivity sufficient to detect from 1 to about 10 copies of a nucleic acid.

In a first embodiment, the invention provides a method for in situ detection of a nucleic acid analyte within a sample of biological material based on bDNA hybridization. The method comprises the steps of (a) preparing at least a portion of a processed patient sample that includes mRNA or cDNA thereof, (b) contacting the biological material with a target oligonucleotide probe under hybridizing conditions, (e) washing the biological material, and (d) detecting any analyte-target probe complex on the substrate.

When the nucleic acid analyte comprises double-stranded DNA, it is necessary to denature the DNA such that probe hybridization may take place. Suitable denaturing steps include exposing the sample to an alkali or heat treatment.

In addition, when the nucleic acid analyte comprises DNA, it is also necessary to digest any RNA that may be present. Generally, any method known in the art for digesting RNA may be used. It is preferred, however, that RNase is used. Alternatively, if the nucleic acid analyte comprises RNA, digestion of RNA is not performed. Heating of messenger RNA (mRNA) may be required, however, to remove secondary structure.

Once prepared, the biological material is placed in contact with a detection probes under hybridizing conditions. The target probe has a portion that is complementary to at least a portion of the target sequence of the nucleic acid analyte.

When the nucleic acid analyte of interest is present in the sample, the nucleic acid analyte and detection probe hybridize to form an analyte-probe complex.

Once a sufficient incubation period has passed, both the substrate and analyte-probe complex, if present, are washed to facilitate the removal of unbound detection probe. The washing step requires the use of a washing fluid that generally comprises a buffer solution and, inter alia, a detergent. The buffer solution may be any conventional solution known in the art suitable for removing unhybridized oligonucleotide probes. Preferred buffer solutions comprise the salts of alkali metals. Particularly preferred buffer solutions comprise sodium chloride, sodium citrate and combinations thereof. The detergent is preferably a non-ionic detergent. In addition, it is preferred that the detergent is also a hydrophilic surfactant. Exemplary detergents are polyoxyethylene-based detergents, e.g., BRIP and TRITON.

Once the washing fluid is determined, the washing step is carried out at least one, preferably two, and most preferably three times. It has been found that the temperature of the wash step influences the sensitivity of the assay. Optimally, the wash step is carried out at room temperature.

Once the washing step is complete, unhybridized detection probes are absent. Thus, any method that can detect the analyte-target probe complexes on the substrate may be used to determine the presence of the nucleic acid analyte. It is preferred, however, to add additional oligonucleotide probes corresponding to portions of the analyte sequence which do not basepair with the detection probe, such that a branched network is formed. Once a branched network is formed, a plurality of detectable labels is added with the effect of “amplifying” the signal for facile detection. Thus, detecting an analyte-probe complex can be accomplished by:

(i) contacting the washed substrate and analyte-probe complex with a preamplifier oligonucleotide probe under hybridizing conditions, wherein a first portion of the preamplifier probe is complementary to a portion of the analyte sequence other than the portion that is complementary to the detection probe, thereby forming an analyte-probe-preamplifier probe complex when the nucleic acid analyte is present in the sample;

(ii) contacting the product of step (i) with an amplifier oligonucleotide probe under hybridizing conditions, wherein a first portion of the amplifier probe is complementary to a second portion of the preamplifier probe, thereby forming an analyte-probe-preamplifier probe-amplifier probe complex when the nucleic acid analyte is present in the sample;

(iii) contacting the product of step (ii) with a labeled oligonucleotide probe under hybridizing conditions, wherein a portion of the label probe binds to a second portion of the amplifier probe, thereby forming an analyte-probe-preamplifier probe-amplifier probe-label probe complex when the nucleic acid analyte is present in the sample;

(iv) labeling the analyte-probe-preamplifier probe-amplifier probe-label probe complex with a detectable label; and

(v) detecting the presence of the label on the substrate.

Labeling is accomplished when the label probe hybridizes to the analyte-probe-preamplifier probe-amplifier probe complex. The label probe includes one or more detectable labels that directly or indirectly provide for a detectable signal. The labels maybe bound, covalently or non-covalently, to the label probe as individual members of the complementary sequence, or may be present as a terminal. member or terminal tail having a plurality of labels. Various means for providing labels bound to a probe have been reported in the literature. See, for example, Leary et al. (1983) PNAS 80:4045; Renz et al. (1984) Nuc Acids Res 12:3435; Richardson et al. (1983) Nuc Acids Res 11:6167; Smith et al. (1985) Nuc Acids Res 13:2399; Meinkoth et al. (1984) Anal Biochem 138:267. Labels that may be employed include fluorescers, chemiluminescers, dyes, enzymes, enzyme substrates, enzyme cofactors, enzyme inhibitors, enzyme subunits, metal ions and the like. Illustrative specific labels include fluorescein, rhodamine, Texas red, phycoerythrin, luminol, NADPH, horseradishperoxidase, and alkaline phosphatase, among others.

Detection of the detectable label can be accomplished by any art-known means and is dependent upon the nature of the label. For fluorescers, a large number of fluorometers are available. For chemiluminescers, luminometers or films are available. With enzymes, a fluorescent, chemiluminescent, or colored product can be provided and determined fluorometrically, luminometrically, spectrophotometrically or visually (preferably with the aid of a microscope). For the present method, it is preferred that the alkaline phosphatase substrate is added to detect the presence of the alkaline phosphatase label using bright field or fluorescence microscopy.

IV. Additional Applications

The utility of the invention is not limited to diagnosis. The system and methods described herein may also be useful for screening, making prognosis of disease outcomes, and providing treatment modality suggestion based on the profiling of the pathologic cells, prognosis of the outcome of a normal lesion and susceptibility of lesions to malignant transformation.

The utility of the present invention is also not limited to the detection of oral cancer. The systems and methods described herein may also find utility in detecting DNA mutations in following conditions, including but not limited to, Orofacial Clefts, Crouzon Syndrome (Craniofacial Dysostosis), Apert Syndrome (Acrocephalosyndactyly), Treacher Collins Syndrome, and Amelogenesis Imperfecta.

In addition, the systems and methods described herein may also be useful in detecting abnormal RNA transcripts and/or protein products in following conditions, including but not limited to, all epithelial pathologies including simple cysts, all salivary gland pathologies, all soft tissue tumors, all bone pathologies and all systemic diseases with oral manifestations (for example, diabetes).

The invention may find additional utility in diagnosing multiple forms of infections, including bacterial infection, fungal and protozoal infection and viral infection. Bacterial infections include, but not limited to, Acute Necrotizing Ulcerative Gingivitis, Impetigo, Erysipelas, Streptococcus, Scarlet Fever, Tonsilolithiasis, Diptheria, Syphilis, Gonorrhea, Tuberculosis, Leprosy (Hansen's Disease), NOMA (Cancrum Oris; Gangrenous Stomatitis; Necrotizing Tomatitis), Actinomycosis, Cat Scratch Disease. Fungal and protozoal infections include, but not limited to, Candidiasis, Histoplasmosis, Blastomycosis, Paracoccidiomycosis, Coccidiomycosis, Cryptococcosis, Zygormycosis, Aspergillosis, and Toxoplasmosis. Viral infections include, but not limited to, Herpes Simplex, Varicella, Herpes Zoster, Infectious Mononucleosis, Cytomegalovirus, Enteroviruses, Rubeola, Rubella, Mumps and HIV.

V. Illustrative Embodiments

To provide an overall understanding of the invention, certain illustrative embodiments will now be described. However, it will be understood by one of ordinary skill in the art that the systems and methods described herein can be adapted and modified for other suitable applications and that such other additions and modifications will not depart from the scope hereof. For example, it is contemplated that the systems and methods described herein are also useful for screening, prognosis of disease outcomes, and providing treatment modality suggestion based on the profiling of the pathologic cells, prognosis of the outcome of a normal lesion and susceptibility of lesions to malignant transformation.

The systems and methods of the invention include methods for allowing a patient-care physician, a dentist, a medical technician, a nursing practitioner, or some other medical professional to take a sample from a patient and, at the point of sample collection, run a diagnostic assay on that sample to determine whether the patient tests positive or negative for a particular indication. More particularly, the systems and methods of the invention are understood to include a desktop system that provides a self-contained diagnostic or screening tool for processing a prepared sample to determine whether a dental patient tests positive for the indication of oral cancer.

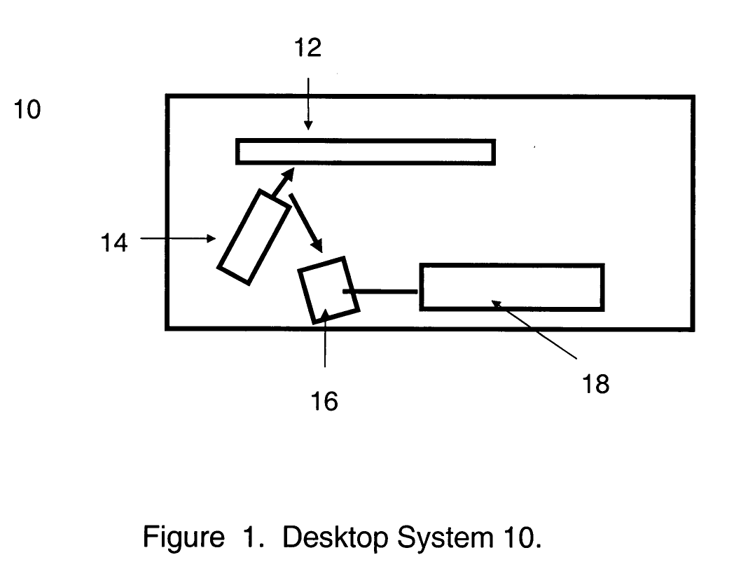

To this end, the invention may include in one embodiment a desktop system, such as the desktop system 10 depicted in FIG. 1. As shown in FIG. 1, the desktop system 10 may include a defractive grating surface that has adhered to its surface a biological compound which is capable of hybridizing with certain compounds. As further shown by FIG. 1, the system 10 may include a light source 14 that in one embodiment, provides a source of laser light that may be directed at the back surface of the substrate 12. Light from the back surface of the substrate 12 may be reflected back onto the optical sensor 16. The optical sensor 16 is of the type capable of detecting a wavelength shift within the reflected light. The signal detected by the device 16 may be communicated to the computer system 18. The depicted computer system 18 may be capable of processing information from detector 16 to determine the meaning and significance of the spectral shifts measured by the detector 16.

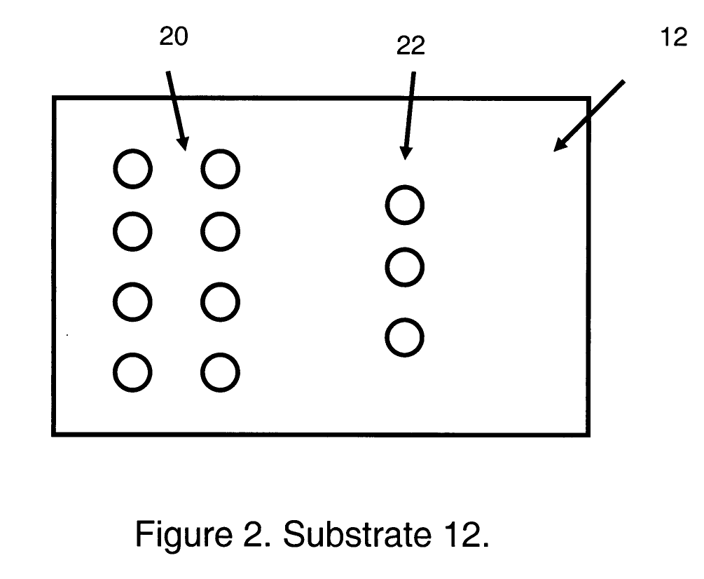

In operation, the system 10 detects molecular interactions by measuring the spectral shift occurring at locations that are mapped to certain probes. Thus the substrate 12 may be a diffractive grating that is employed as a surface binding platform. When illuminated with white light, the substrate 12 is designed to reflect only a single wavelength. When molecules are attached to the surface, the reflected wavelength is shifted due to the change of the optical path of light that is coupled into the grating. FIG. 2 depicts the substrate 12 having probes attached to its surface. The probes can include actual probes and control probes. By linking probes or other receptor molecules to the surface of substrate 12, complementary binding molecules can be detected within the sample without the use of fluorescent probes or particle labels. It is understood that the detection technique is capable of resolving changes of ˜0.1 nm thickness of protein binding, and can be performed with the grating surface either immersed in fluid or dried.

The readout system consists of the optical detector 16 that collects reflected light. A single spectrometer reading may be performed for each location of interest. The reading may occur in several milliseconds, thus making it possible to quickly measure a large number of molecular interactions taking place in parallel upon a grating surface, and to monitor reaction kinetics in real time, or near real-time. Thus the system 10 provides a desktop diagnostic device that can allow a medical professional, such as a dentist or hygienist, to perform a real-time diagnosis of an indication such as oral cancer.

In one embodiment, the system 10 allows for real-time analysis of gene expression. In the case of oral cancer, the system 10 may monitor for the expression characteristics of 45 genes that have been discovered to be strongly correlated with epithelial cancer, and particularly oral tumor malignancy. The elevated expression of three of these genes was further confirmed by real-time quantitative PCR of the original samples as well as samples from five new pairs of cases. Of the 45 genes identified, 6 have been previously implicated in the disease, and 2 are uncharacterized clones. The present invention provides the ability to analyze changes in the levels of the transcripts and/or protein products for multiple different genes in oral or other epithelial tissue.

In a further embodiment, the system 10 may find utility in the detection of a malignant condition in cell specimens from the cervix, vagina, uterus, bronchus, prostate, gastrointestinal tract including oral pharynx, mouth, etc., and exfoliative cell specimens taken from impressions of the surface of tumors or cysts, the cut surface of biopsy specimens, especially lymph nodes, and serous fluids.

Also depicted in FIG. 1 is a data processing system capable of processing the spectral data collected by the system 10. The data processing system can comprise any suitable device and in one embodiment is a single board computer system that has been integrated into a system 10 for analyising the spectral data and comparing that data to expression profile information that has been classified as indicating the presence or absence of a particular condition. The single board computer (SBC) system can be any suitable SBC, including the SBCs sold by the Micro/Sys Company, which include microprocessors, data memory and program memory, as well as expandable bus configurations and an on-board operating system.

As discussed above, the system 10 may execute a software process that analyzes the spectral data in real-time. The software for performing such an analysis may be implemented as a C language computer program, or a computer program written in any high level language including C++, Fortran, Java or basic. Additionally, in an embodiment where microcontrollers or DSPs are employed, the software can be realized as a computer program written in microcode or written in a high level language and compiled down to microcode that can be executed on the platform employed. The development of such programs is known to those of skill in the art, and such techniques are set forth in Digital Signal Processing Applications with the TMS320 Family, Volumes I, II, and III, Texas Instruments (1990). Additionally, general techniques for high level programming are known, and set forth in, for example, Stephen G. Kochan, Programming in C, Hayden Publishing (1983). It is noted that DSPs are particularly suited for implementing signal processing functions, including preprocessing functions such as image enhancement through adjustments in contrast, edge definition and brightness. Developing code for the DSP and microcontroller systems follows from principles well known in the art.

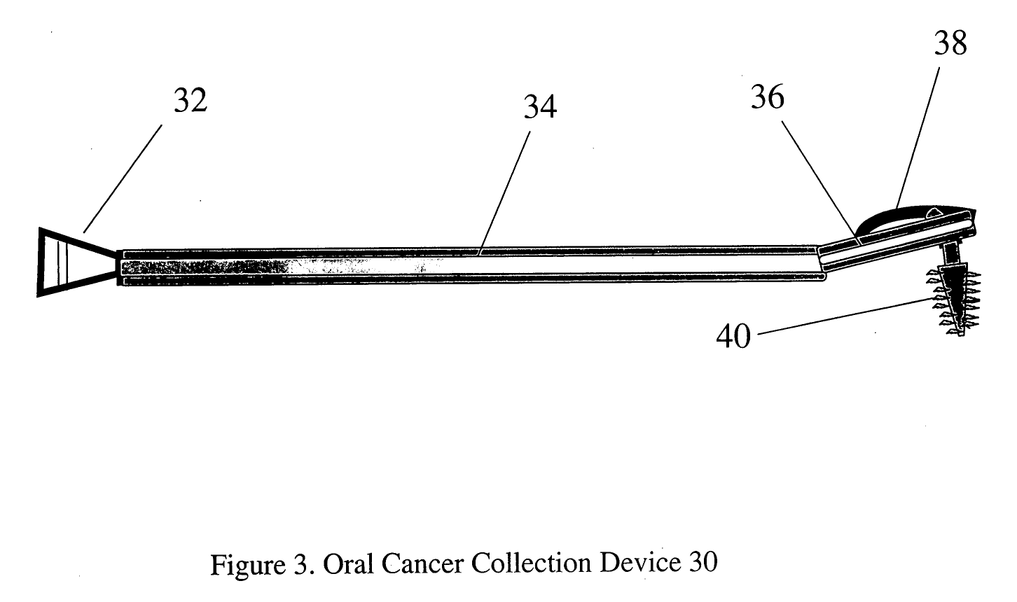

The system also includes in one embodiment a collection device, such as the oral cancer collection device 30 depicted in FIG. 3. As shown in FIG. 3, the collection device 30 may include a triangular collection tube 32, a large luminal tube 34, a small luminal tube 36. In operation, the collection device 30 collects saliva samples from a patient by placing the small luminal tube 36 into the mouth of a patient to collect saliva. The saliva samples collected pass through the small luminal tube 36, and continue through the large luminal tube 34 to reach the triangular collection tube 32. The saliva sample so collected can be subsequently used for further screenings as discussed in previous sections of this application. Thus, the collection device 30 allows a dentist or a hygienist to perform sample collection in a quick and convenient fashion during a regular dental office visit.

In an alternative embodiment, the collection device 30 may include additionally a lock 38 and a sampling brush 40 for the collection of cell or tissue samples from patients. In operation, the sampling brush 40 can be used to collect cell or tissue samples and is locked in place by the lock 38.

The collection device may be made of metal, plastic, polypropylene, polyethylene, various thermal elastomers, and other engineering polymers/plastics or other materials facilitating normal handling and cleaning for dental devices. Furthermore, the material used may be rigid, semi-rigid, or elastic.

Those skilled in the art will know or be able to ascertain using no more than routine experimentation, many equivalents to the embodiments and practices described herein. Accordingly, it will be understood that the invention is not to be limited to the embodiments disclosed herein, but is to be understood from the following claims, which are to be interpreted as broadly as allowed under the law.

Exemplification

To help elucidate the genetic and biochemical mechanisms underlying the onset of oral epithelium cancer, the expression phenotype (transcriptome) of oral epithelium was probed using expression microarrays, specifically the Affymetrix HuGeneFL® microarray containing ˜7000 human genes. The accuracy of the measured expression levels has been assessed to be approximately 82% (3, 4), so that meaningful gene induction and repression differences can be thus monitored. Although microarrays provide a vast amount of information about the state of transcription in cells and tissues, they must be complemented by appropriate bioinformatic methods for the extraction of useful biological knowledge and the overall upgrade of their information content. We illustrate below the application of two such methods and have succeeded in identifying 45 genes that are strongly correlated with the appearance of malignancy in oral epithelium. The importance of these findings stems from the implication of associated genetic and biochemical mechanisms in oral carcinogenesis that may lead to the definition of new targets for the development of diagnostic tools and therapeutic procedures.

Samples were obtained from 5 patients with oral cancer and immediately snap frozen. Laser capture microdissection (LCM) was used to procure malignant and normal oral keratinocytes. LCM, RNA isolation, T7 linear amplification, probe biotinylation, GeneChip® array hybridization and subsequent scanning were applied as previously described (3, 4). Array to array reproducibility was determined by comparing the signals from duplicate microarrays as well as n-tuplicate features on the same microarray. Differences in expression equivalent to less than one copy per cell were detected for 24 transcripts at >99% confidence (p<0.01, unpaired t-test). Copies per cell are calculated using known concentrations of control transcripts, assuming an average transcript length of 1 kb and a population of 300,000 transcripts per cell.

There are several research issues that can be addressed with microarray data, each requiring a particular set of bioinformatic tools. Commonly asked questions include: (a) Of the large number of genes probed, which ones are particularly relevant to a disease or, in general, a cellular state of interest, by virtue of their ability to characterize a particular cellular state as such; (b) Is there a specific pattern of gene expression that marks the occurrence of a particular physiological state; and (c) Can such patterns be used to diagnose the physiological state of cell and tissue samples. Although some answers to the above questions can be obtained by simple visual inspection of a sample's expression levels relative to those of the control, statistical significance is increased by using multiple samples from each class and applying rigorous analysis in identifying discriminatory genes and their characteristic patterns.

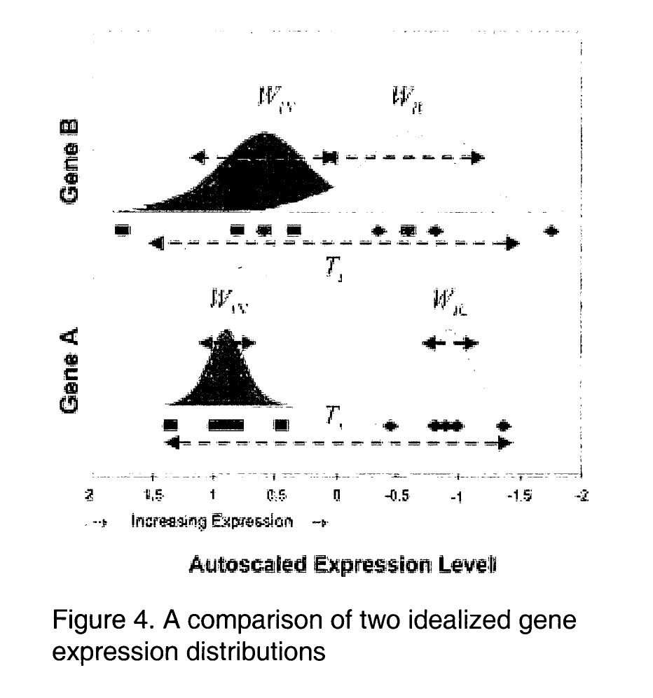

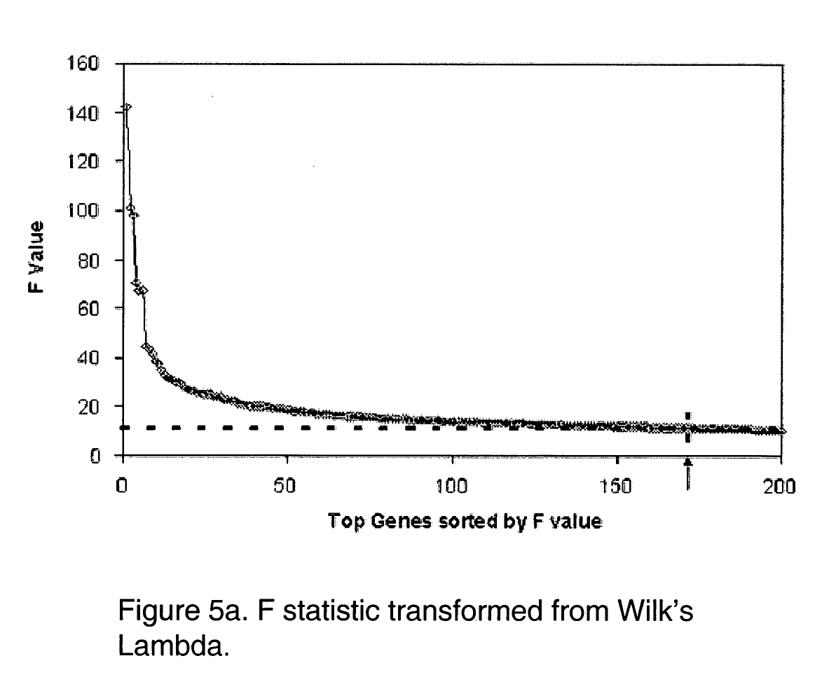

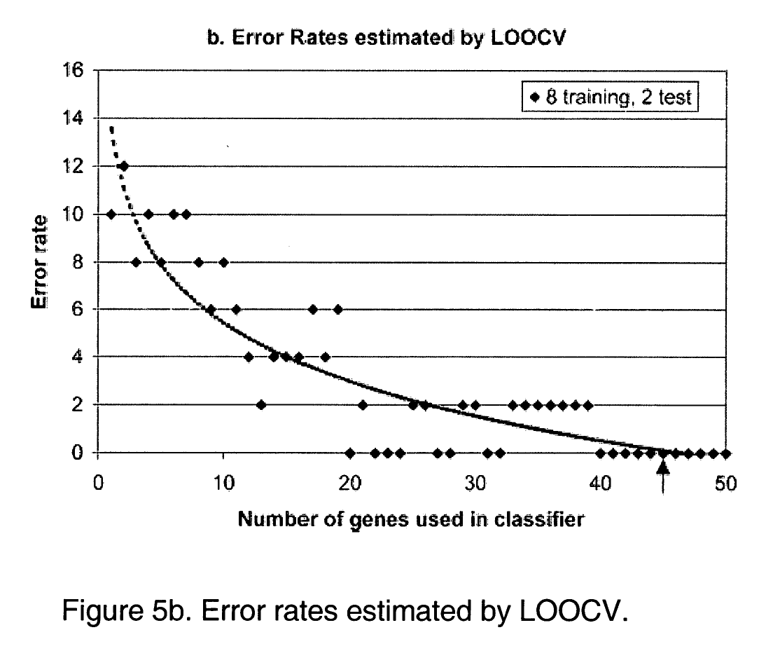

It is expected that only a subset of the total number of genes probed by microarrays will be of consequence in distinguishing a physiological state of interest. This is shown schematically in FIG. 4 depicting the expression distribution of two genes A and B in ten samples obtained from two different types (or classes) of tissues, such as normal and diseased. Clearly, while the expression of gene A is sufficiently distinct in the two types, the significant overlap in the expression of gene B for the two classes of samples reduces its value in differentiating one class of tissue from another. As shown in FIG. 4, the ratio of the “within group variance” to the “total variance” (also known as the Wilks' lambda score, 5) can be used as a metric of each gene's class differentiating potential. Since Wilks' lambda score does not follow any known distribution, the transformation shown in FIG. 5a is applied to approximate Wilks' lambda ratio by a univariate F statistic that allows one to identify discriminatory genes with a specified statistical significance. By this approach, 171 genes are identified whose between-group-variance is significantly larger, under the level of significance (α=0.01), than the variance when the ten samples are considered as a single group.

A stronger classification criterion can be obtained by using the error classification rate (5, 6, 7). In this method, a subset of the available samples (the training set) is used to identify the discriminating genes as well as to define a sample classification model. The classification model (see below) is subsequently tested against the samples that were not included in the training set (the test set) and the misclassification rate is calculated for all possible membership configurations of the training and test sets. This procedure is initiated with a classifier that is based on a single (most discriminating) gene and is repeated as more genes (in order of discriminating power based on their F value) are added to the classifier. The misclassification rate would be expected to decrease as more and more genes are added to the classifier, making it more robust. This is exactly what is observed with the expression data of oral epithelium cancer, as shown in FIG. 5b. Clearly, 40-45 genes are sufficient to accurately predict the class of the samples in the test set and, as such, they are deemed most discriminatory of the oral epithelium cancerous state.

The misclassification rate is a function of both the sample population size and the number of genes considered. Even with only three samples describing each of the two states (that is, reserving 2 of the 5 samples to test the classifier developed using the other 3), correct classification is achieved over 85% of the time if a sufficient number of genes are considered. Four samples from each group (leave one out case) were sufficient to achieve perfect classification for all permutations of the training and testing sets when at least 45 genes are considered. These results show that accurate classification can be achieved even with only a few samples if a sufficient number of genes are included in the classifier.

Table 1 summarizes the discriminatory genes obtained by applying the above procedure to the oral epithelium gene expression data. As an additional validation step of the experimental and computational methods used in deriving these results, we selected three genes from Table 1 whose expressions are consistently altered in the 5 paired cases of oral cancer and applied real-time quantitative PCR (RT-QPCR) to independently measure their expression levels. The three genes were Neuromedin U (interacting protein with G-protein coupled receptors), Wilm's tumor related protein (tumor suppressor) and aldehyde dehydrogenase-10 (xenobiotic enzyme, fatty aldehyde dehydrogenase). Table 2 summarizes the RT-QPCR results of these three genes in the original 5 cases as well as 5 new independent cases of oral cancer. For the three genes identified, a positive comparison between the GeneChip® expression data and RT-QPCR data is observed for more than 80% of the cases examined (3).

Besides expression differences in individual genes for the two types of tissues, discriminating genes can also be used collectively to define a composite index of cell physiology, using Canonical Discriminant Analysis (CDA) (8). CDA defines a new projection space of lower dimensions where the “between class” variance of the various class samples is maximized. The projection space is defined by Canonical Variables (CV) that are linear combinations of the individual gene expressions, much in the same way as Principal Component Analysis (PCA) (9) and Singular Value Decomposition (SVD) (10, 11) define a projection space where the total variance of the sample points is maximized. Both CDA and PCA use the same eigenvalue decomposition procedure to define the linear projection (5); however, their objective functions are different. By maximizing the between group variance and minimizing the within group variance, CDA generates new projection variables (CV) along which the “between group” variance relative to the “within group” variance is maximized. This allows samples of different predefined classes to cluster in distinct areas of the projection space. In cases where the classes are known a priori, the resulting CV's have much more biologically relevant information than principal components calculated through undirected application of PCA.

Applying the CDA projection to the expression data from the oral epithelium tissues yielded the two distinct classes, each of them characteristic of the physiological states of normal and malignant oral epithelium. Consequently, the linear combinations of expression data reflected in the canonical variables represent composite metrics that define distinctly the expression phenotype of the corresponding physiological states. These phenotypes, in turn, can be used to classify unknown samples using the expression profiles of the differentiating genes. The classifier employed in the development of the algorithm of FIG. 5 assigned samples to a particular class based on their distance from the mean of the class in the CDA projection space. The reliability of the classification power provided by expression analysis has already been shown in FIG. 5.

Discussion of Discriminatory Gene Results

The 45 genes identified by the previous classification schemes exhibit close association with oral cancer development. Two thirds (30) of the genes are downregulated in cancer while ⅓ (15) of the genes are upregulated in cancer. Six of these genes (13%) have been associated with oral cancer either in previous literature (urokinase plasminogen activator (12,13), cathepsin L (12,14) cytochrome P450 (15), ferritin light polypeptide (16), interleukin 8 receptor beta (17)), or by association with chromosomal aberrations found in oral cancers (phospholipase A2). For 39 of the 45 discriminating genes identified by our experimental analysis there is no previously reported chromosomal aberration or differential gene expression. Thus our approach may have identified many candidate genes central to the genesis of oral cancers. Table 1 shows that a number of these genes are members of biological and functional pathways important to tumorigenesis: metastasis and invasion (urokinase plasminogen activator, oncofetal trophoblast glycoprotein, cathepsin L, Wilms tumor related protein, FAT); oncogenes (GRO2, AML1); tumor suppressors (Wilms tumor related protein, FAT); cell cycle and related proteins (heat shock protein 90); signal transducers (crystallin alpha-B) and members of xenobiotic metabolism pathways (aldehyde dehydrogenase-9, aldehyde dehydrogenase-10, carboxylesterase-2, cytochrome p450).

An objective of this study is to identify genes not previously implicated in cancer and place them into functional pathways or to identify genes with diagnostic and predictive value. The outcome of the study provides data which can generate testable hypotheses. The differentially expressed genes that are not yet functionally characterized or associated in head and neck/oral carcinogenesis are examined. Neuromedin U (Nmu) is significantly downregulated in 5/5 oral tumors examined. Nmu is a less understood protein that manifests potent contractile activities on smooth muscle cells (18). Recently, two G-protein coupled receptors (Nmu1 and Nmu2) have been identified to interact with Nmu with nanomolar potency (19). The data provide strong evidence that Nmu is relevant in the development of oral malignancy and suggest the need for further study of the role of Nmu (down regulated expression in tumor) in carcinogenesis.

One finding is the homology of the translocase of outer mitochondrial membrane 34 (TOM34) with the Drosophila melanogaster Hsp70/Hsp90 organizing protein homolog (AF056198). Both TOM34 and Heat Shock 90 Kd (Hsp90) are in the discriminatory gene list and both are upregulated in cancer. Also upregulated in cancer is Heat Shock protein 70 Kd (Hsp70) which is also ranked high in the discriminatory list although it did not make it to the top 45 genes (ranked at #88, well within the α=0.01 confidence limit used in considering the Wilks' lambda criteria). Several cellular signaling proteins require the coordinated activities of the two heat shock proteins Hsp70 and Hsp90 for their folding, oligomeric assembly and translocation. These substrates include several proto-oncogenic serine, threonine and tyrosine kinases such as Raf and Src (20). Hsp90 is essential for Raf function in vivo (21). Another member of this pathway found in the discriminatory gene list is Lymphocyte Cytosolic Protein 2 (rank #***, again within the α=0.01 confidence limit) (SLP76), (U20158). SLP76 associates with Grb2 adaptor protein and is a substrate for phosphorylation. The concurrent upregulation of TOM34, Hsp90 and Hsp70 and SLP 76 in cancer suggests upregulation of the signal transduction pathway. Interestingly, our analysis identified a tyrosine receptor kinase (HER3), as well as a secreted protein that activates a tyrosine receptor kinase (FGF8), downregulated in the cancer cells. Further studies are needed to deduce which ligand or ligands and which tyrosine kinase receptors are responsible for the hyperfunctional signal transduction pathways.

One of the hallmarks of oral cancer is the decreased host immune reaction to the tumor. We found downregulation of MHC class I polypeptide-related sequence A, (MICA). Receptors for MICA have been identified in many types of T cells, as well as natural killer (NK) cells. In our analysis MICA is downregulated in the tumor samples, suggesting a negative modulation of the immune response against the transformed cells (22).

The discriminatory gene list also reveals a number of known genes, such as HER3 and FAT, that are expressed contrary to tumors at other anatomical sites. This work indicates how bioinformatic analysis of micro array expression data can generate specific hypotheses to be further tested by specifically designed experiments. Such hypotheses are data driven and, as such, define a new approach to scientific research.

A subset of these 45 genes useful for oral cancer diagnosis may be further identified by applying the statistical methods described above to oral cancer samples and normal samples. The expression profile of the subset of genes may provide a more robust disease signature useful for diagnostic and prognosis purposes. For example, the subset of genes may be less than 20 genes, less than 10 genes, or less than 5 genes.

One skilled in the art readily appreciates that the present invention is well adapted to carry out the objects and obtain the ends and advantages mentioned, as well as those inherent therein. The methods, systems and kits are representative of preferred embodiments, are exemplary, and are not intended as limitations on the scope of the invention. Modifications therein and other uses will occur to those skilled in the art. These modifications are encompassed within the spirit of the invention and are defined by the scope of the claims. It will be readily apparent to a person skilled in the art that varying substitutions and modifications may be made to the invention disclosed herein without departing from the scope and spirit of the invention.

The practice of the present invention will employ, unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999. All patents, patent applications and references cited herein are incorporated in their entirety by reference.

References

1. E. E. Vokes, R. R. Weichselbaum, S. M. Lippman, W. K. Hong, New England Journal of Medicine 328, 184-94 (1993); D. Sidransky, Curr Opin Oncol 7, 229-33 (1995).

2. S. P. Schantz, Eur Arch Otorhinolaryngol 250, 424-8 (1993); S. P. Schantz, Current Opinion in Oncology 5, 483-90 (1993).

3. I. Alevizos et al., submitted (2000).

4. H. Ohyama et al., Biotechniques 29, 530-6 (2000).

5. R. A. Johnson, D. W. Wichern, Applied Multivariate Statistical Analysis. Prentice Hall, (1992).

6. P. A. Lachenbruch, M. R. Mickey, Technometrics, 10, 1-11, (1968).

7. SAS/STAT User's Guide. SAS Institute Inc., (1989).

8. W. R. Dillon, M. Goldstein, Multivariate Analysis. Wiley, (1984).

9. X. L. Wen et al., Proceedings Of the National Academy Of Sciences Of the United States Of America, 95, 334-339, (1998).

10. N. S. Holter, et al., Proceedings Of the National Academy Of Sciences Of the United States Of America, 97, 8409-8414, (2000).

11. O. Alter, P. O. Brown, D. Botstein, Proceedings Of the National Academy Of Sciences Of the United States Of America, 97, 10101-10106, (2000).

12. H. Kawamata et al., Int J Cancer 70, 120-7 (1997).

13. S. Nozaki et al., Oral Oncol 34, 58-62 (1998).

14. P. Strojan et al., Clin Cancer Res 6, 1052-62 (2000).

15. G. I. Murray et al., Gut 35, 599-603 (1994).

16. C. Leethanakul et al., Oral Oncol, 36(5), 474-83 (2000).

17. B. L. Richards et al., Am J Surg., 174(5), 507-12 (1997).

18. P. G. Szekeres et al., J Biol Chem 275, 20247-50 (2000).

19. R. Fujii et al., J Biol Chem 275, 21068-74 (2000); R. Raddatz et al., J Biol Chem (2000).

20. C. Scheufler et al., Cell 101, 199-210 (2000); D. F. Nathan, S. Lindquist, Mol Cell Biol 15, 3917-25 (1995).

21. A. van der Straten, C. Rommel, B. Dickson, E. Hafen, Embo J 16, 1961-9 (1997).

22. S. Bauer et al., Science 285, 727-9 (1999).

23. P. Madsen et al., J. Biol. Chem. 270, 5823-5829 (1995).

24. B. Carnemolla et al., J. Biol. Chem. 271, 8157-8160 (1996).

TABLE 1
List of 45 discriminatory genes of oral epithelium cancer

| | | Up/Down | | | | |
|---|---|---|---|---|---|---|
| Accession | | Regulated in | Oral Cancer | Chromosome | | |
| Number | Gene Name | Cancer | Association | Location | Function | Significance |
| X76029 | Neuromedin U | Down in cancer | | 4q12 | | unexpected finding |
| U34252 | Aldehyde dehydrogenase 9 | Down in cancer | | 1q22-q23 | xenobiotic | |
| | (Human gamma- | | | | metabolism | |
| | aminobutyraldehyde | | | | | |
| | dehydrogenase E3 isozyme) | | | | | |
| U47011 | Fibroblast growth factor 8 | Down in cancer | | 10q24 | oncogene | opposite result than |
| | | | | | | expected |
| M34309 | Human epidermal growth factor | Down in cancer | | 12q13 | | opposite result than |
| | receptor (HER3) | | | | | expected |
| U58970 | Translocase of outer | Up in cancer | | 20 | | mitochondrial protein |
| | mitochondrial membrane 34 | | | | | import |
| D42047 | KIAA0089 | Down in cancer | | 3 | Related to mouse glycerophosphate | |
| | | | | | dehydrogenase | |
| M69177 | Monoamine oxidase B | Down in cancer | | Xp11.4-p11.3 | | |
| X02419 | Urokinase plasminogen activator | Up in cancer | + | 10q24 | Biomarker | Invasion pathway |
| X78932 | Zinc finger protein 273 | Down in cancer | | N/A | Transcription factor | |
| Z78289 | clone 1D2 | Down in cancer | | N/A | | |
| U46689 | Aldehyde dehydrogenase 10 (fatty | Down in cancer | | 17p11.2 | Xenobiotic | |
| | aldehyde dehydrogenase) | | | | metabolism | |
| Y09616 | Carboxylesterase 2 (intestine, liver) | Down in cancer | | 16 | xenobiotic | |
| | | | | | metabolism | |
| M57731 | Gro2 oncogene | Up in cancer | | 4q21 | 90% identical to Gro1 | |
| M14200 | Diazepam binding inhibitor | Down in cancer | | 2q12-q21 | | |
| U07969 | Cadherin 17 | Down in cancer | | 8q22.2-q22.3 | Cadherin family | In a chromosomal location |
| | | | | | | where LOH is |
| | | | | | | present |
| M74558 | TAL1 (SCL) interrupting locus | Up in cancer | | 1q32 | SCL interrupting | leukemia associated gene |
| | | | | | locus | |
| S45630 | Crystallin alpha B | Down in cancer | | 11q22.3-q23.1 | molecular chaperone | small heat shock protein |
| | | | | | activity | |
| Z29083 | 5T4 oncofetal trophoblast | Up in cancer | | 6 | Metastasis | contributes to the |
| | glycoprotein | | | | | process of placentation |
| | | | | | | or metastasis by |
| | | | | | | modulating cell |
| | | | | | | adhesion, shape and |
| | | | | | | motility |
| U56814 | Deoxyribonuclease I-like 3 | Down in cancer | | 3p21.1-3p14.3 | apoptosis related | |
| X15183 | Heat-shock protein 90-kDa | Up in cancer | | 1q21.2-q22 | | |
| U59919 | Smg GDS-associated protein | Up in cancer | | 1 | phosphorylated by v-src | signal transduction |
| | | | | | | pathway |
| M19961 | Cytochrome c oxidase | Down in cancer | | 2cen-q13 | | |
| | subunit Vb (coxVb) | | | | | |
| HG3549- | Wilm Tumor-Related Protein | Down in cancer | | N/A | | |
| HT3751 | | | | | | |
| U18934 | TYRO3 protein tyrosine kinase | Down in cancer | | 15q15.1-q21.1 | | |
| X87241 | FAT tumor suppressor | Up in cancer | | 4q34-q35 | tumor suppressor | opposite results |
| J04469 | Creatine kinase, mitochondrial 1 | Down in cancer | | 15q15 | | |
| M11147 | Ferritin, light polypeptide | Up in cancer | + | 19q13.3-q13.4 | biomarker (16) | |
| U19345 | Transcription factor 20 | Down in cancer | | 22q13.2-q13.3 | Metastatic pathway | controls stromelysin |
| | | | | | | expression |
| L14848 | MHC class I polypeptide related | Down in cancer | | 6p21.3 | | |
| | sequence A | | | | | |
| D13643 | KIAA0018 gene product 1 | Down in cancer | | 1 | | |
| U06643 | Lectin galactoside-binding, | Down in cancer | | 19 | role in cell-cell and/or cell-matrix interactions | |
| | soluble, 7 (galectin 7) | | | | necessary for normal growth control (23) | |
| X98085 | Tenascin-R (restrictin, janusin) | Down in cancer | | 1q24 | contains EGF-like repeats(24) | |
| M28825 | CD1A antigen, a polypeptide | Down in cancer | | 1q22-q23 | | |
| M61855 | Cytochrome P4502C9 subfamily | Down in cancer | + | 10q24 | xenobiotic | |
| | IIC (mephytoin4-hydroxylase), | | | | metabolism | |
| | polypeptide 9 | | | | | |
| U24577 | Phospholipase A2, group VII | Up in cancer | + | 6p21.2-p12 | | |
| HG2992- | Beta-Hexosaminidase, Alpha | Up in cancer | | N/A | | |
| HT5186 | Polypeptide, Abnormal Splice | | | | | |
| | Mutation | | | | | |
| Z78285 | clone 1A7 | Up in cancer | | N/A | | |
| D79994 | KIAA0172 gene | Down in cancer | | 9 | | |
| L19593 | Interleukin 8 receptor, beta | Down in cancer | + | 2q35 | (17) | |
| M30818 | Myxovirus (influenza) resistance 2, | Up in cancer | | 21q22.3 | | |
| | homolog of murine | | | | | |
| U67963 | Lysophospholipase like | Down in cancer | | 3 | | |
| U11877 | Interleukin-8 receptor type B, | Down in cancer | | | | |
| | splice variant IL8RB9 | | | | | |
| X07695 | keratin 4 | Down in cancer | | 12q13 | | |
| D43968 | Runt-related transcription factor | Up in cancer | | 21q22.3 | transcription factor | |
| X12451 | Cathepsin L | Up in cancer | + | 9q21-q22 | Metastasis | |

TABLE 2
Validation of 3 discriminatory genes (identified by
GeneChip ® profiling and bioinformatic analysis)
by real-time quantitative PCR (RT-QPCR). Shown are the
numbers of cases where statistically significant differences
between the control and malignant samples were found
in the expression levels of the indicated genes using the
two methods. GC = GeneChip ® data.

| | Neuromedin U | | WT-1 | | ALDH-10 | |
|---|---|---|---|---|---|---|
| | GC | RT-QPCR | GC | RT-QPCR | GC | RT-QPCR |
| Original 5 | 5/5 | 5/5 | 5/5 | 4/5 | 5/5 | 4/5 |
| Cases | | | | | | |
| 5 New | | 4/5 | | 4/5 | | 5/5 |
| Independent | | | | | | |
| Cases | | | | | | |

1. A method for diagnosing an oral disease in a patient, comprising:
a. obtaining a biological sample from a patient at the point of care;
b. determining the expression level of a plurality of genes associated with an oral disease in the biological sample, thereby producing a test expression profile;
c. comparing the test expression profile with at least one signature expression profile of the plurality of genes indicative of an oral disease;
wherein if the test expression profile substantially matches a signature expression profile indicative of an oral disease, the patient has the oral disease.

2. The method of claim 1, wherein the expression level is protein expression level.

3. The method of claim 2, wherein proteins are isolated from the biological sample before their expression levels are determined.

4. The method of claim 2 or 3, wherein the protein level is determined by a method selected from: immunoassay, protein array and single molecule detection.

5. The method of claim 1, wherein the expression level is mRNA expression level.

6. The method of claim 5, wherein nucleic acids are isolated from the biological sample before their expression levels are determined.

7. The method of claim 5 or 6, wherein the mRNA level is determined by a method selected from: microarray analysis, multiplex PCR analysis and single molecule detection.

8. The method of claim 1, wherein the oral disease is oral cancer and the plurality of genes are the 45 genes of which a signature expression profile is indicative of oral cancer.

9. The method of claim 1, wherein the oral disease is oral cancer and the plurality of genes are a subset of the 45 genes of which a signature expression profile is indicative of oral cancer.

10. The method of claim 1, wherein the biological sample is selected from: saliva, tissue, bone marrow aspirates, bone marrow biopsies, lymph node aspirates, lymph node biopsies, serum, and fine needle aspirates.

11. The method of claim 1, wherein the oral disease includes oral cancer, HIV, tooth decay, gingivitis, pyorrhea, and periodontitis.

12. A method of allowing a dentist to provide for detection of oral disease at the point of patient care, comprising:
a. obtaining a biological sample from a dental patient at the point of care;
b. determining the expression level of a plurality of genes associated with an oral disease in the biological sample, thereby producing a test expression profile;
c. comparing the test expression profile with at least one signature expression profile of the plurality of genes indicative of an oral disease; and
d. notifying the patient the results of the test.

13. The method of claim 12, wherein the expression level is protein expression level.

14. The method of claim 13, wherein proteins are isolated from the biological sample before their expression levels are determined.

15. The method of claim 14, wherein the expression level is mRNA expression level.

16. The method of claim 15, wherein nucleic acids are isolated from the biological sample before their expression levels are determined.

17. The method of claim 15 or 16, wherein the mRNA level is determined by a method selected from: microarray analysis, multiplex PCR analysis and single molecule detection.

18. The method of claim 12, wherein the oral disease is oral cancer and the plurality of genes are 45 genes of which a signature expression profile is indicative of oral cancer.

19. The method of claim 12, wherein the oral disease is oral cancer and the plurality of genes are a subset of the 45 genes of which a signature expression profile is indicative of oral cancer.

20. The method of claim 12, wherein the sample is selected from: saliva, tissue, bone marrow aspirates, bone marrow biopsies, lymph node aspirates, lymph node biopsies, serum, and fine needle aspirates.

21. The method of claim 12, wherein the oral disease includes oral cancer, HIV, tooth decay, gingivitis, pyorrhea, and periodontitis.

22. The method of claim 12, wherein comparing determined expression levels includes
allowing the dentist to select from among a plurality of groups of known oral diseases.

23. The method of claim 12, further including obtaining authorization representative of insurance coverage.

24. The method of claim 23, further comprising selecting a test for an oral disease as a function of insurance coverage.

25. The method of claim 24, further comprising requesting insurance reimbursement for the test.

26. The method of claim 25, further comprising generating a medical record representative of the test and result.

27. A system for allowing a dentist to test for an oral disease at the point of care, comprising:
a. a sample collection device for collecting a sample from a dental patient at the point of care;
b. a diagnostic system for generating a test expression profile by determining the expression level of a plurality of genes associated with an oral disease in the sample, and comparing the test expression profile with at least one signature expression signatures profile representative of an oral disease; and
c. a notification system for notifying the patient the results of the test.

28. The system of claim 27, wherein the diagnostic system comprises a micro-fluidic processing system for determining the expression level of a plurality of genes associated with an oral disease in the sample.

29. The system of claim 28, further comprising means for allowing the dentist to select from a group of tests associated with oral diseases.

30. The system of claim 29, wherein the oral disease is oral cancer.

31. An oral disease detection kit, comprising:
a. a sample collection device for collecting a sample from a patient at the point of care; and
b. a sample delivery device, adapted for use with the system of claim 27 and being capable of delivering the sample into the system to thereby examine the sample for an indication of an oral disease.

32. The kit of claim 30, wherein the oral disease is oral cancer.
